(19) United States
(12) Patent Application Publication
(10) Pub. No.: US 2026/0277327 A1
(43) Pub. Date: Sep. 17, 2026

(54) CONTEXT-AWARE NEURAL SIGNAL DECODING

(71) Applicant: Synchron Australia Pty Limited, ST KILDA EAST (AU)

(72) Inventors: Thomas James OXLEY, Dover, DE (US); Sadegh EBRAHIMI, Dover, DE (US); Shreyas AGRAWAL, Dover, DE (US); Evan SCHNELL, Dover, DE (US); Nicholas F. HARDY, Dover, DE (US)

(21) Appl. No.: 19/565,937

(22) Filed: Mar. 13, 2026

Related U.S. Application Data
(60) Provisional application No. 63/772,712, filed on Mar. 16, 2025.

(51) Int. Cl. G06F 3/01; G06F 3/04812

(52) U.S. Cl. CPC G06F 3/015; G06F 3/04812

(57) ABSTRACT

Neural signals may be decoded to generate control outputs allowing a user to interact with a user interface. The decoding of neural signals may be adapted based on received user interface context information, enabling accurate and responsive control of the user interface. User interface state metadata may be received from the device presenting the user interface and used to generate control output for the device based on features of the neural signals. A signal quality metric may be generated based on the neural signals. The control output and signal quality metric may be transmitted to the device as standard input events. Neurofeedback may be presented based on the signal quality metric, allowing users to adjust their intent and improve control.

### CROSS-REFERENCE TO RELATED APPLICATION

This application claims priority to United States Provisional Patent Application No. 63/772,712, filed Mar. 16, 2025, and titled “BCI-OS COMMUNICATION SYSTEM WITH ADAPTIVE BLUETOOTH HID INTEGRATION,” which is hereby incorporated by reference in its entirety.

### BACKGROUND

Computing systems may allow a user to interact with a user interface through inputs generated by a separate human interface device (HID). The HID may communicate with the computing system to provide an input using a pre-defined protocol, such as a HID protocol. Existing HID protocols may be unidirectional, such that the computing system receives information from the HID, but the HID does not receive information about the computing system. Providing for interaction with new types of human interface devices can present various challenges for existing computing systems.

### SUMMARY

In some aspects, the techniques described herein relate to a computer-implemented method for user interface state based neural control using a brain-computer interface, including: receiving, from an interactive computing device, user interface (UI) state metadata representing an aspect of a UI of the interactive computing device; capturing neural signals from a user with a brain-computer interface; generating decoder input including neural feature data representing features of the neural signals and UI state feature data representing features of the UI state metadata; processing the decoder input to generate a control output using a model configured to jointly condition the control output on the features of the neural signals and the features of the UI state metadata; and transmitting, to the interactive computing device, the control output as a standard input event of the interactive computing device.

In some aspects, the techniques described herein relate to a computer-implemented method, further including generating the neural feature data as a neural feature vector representing a user’s current neural state over a defined time window.

In some aspects, the techniques described herein relate to a computer-implemented method, further including generating the UI state feature data as a context embedding representing the aspect of the UI of the interactive computing device.

In some aspects, the techniques described herein relate to a computer-implemented method, wherein generating the decoder input includes concatenating a neural feature vector and a context embedding.

In some aspects, the techniques described herein relate to a computer-implemented method, wherein receiving the UI state metadata includes receiving data representing at least one of: an identifier of a currently focused UI element, an identifier of an active application, an interaction modality indicator, pointer position data, or an interaction timing parameter.

In some aspects, the techniques described herein relate to a computer-implemented method for user interface state based neural control using a brain-computer interface, including: receiving, from an interactive computing device, user interface (UI) state metadata representing an aspect of a UI of the interactive computing device; capturing neural signals from a user with a brain-computer interface; generating, using a decoder model and neural feature data representing features of the neural signals, action probability data representing probabilities for each of a plurality of candidate actions intended by the user; processing the action probability data and the UI state metadata to generate control output representing a candidate action of the plurality of candidate actions; and transmitting, to the interactive computing device, the control output as a standard input event of the interactive computing device to perform the candidate action.

In some aspects, the techniques described herein relate to a computer-implemented method, wherein processing the action probability data includes adjusting one or more probabilities in the action probability data based on the UI state metadata.

In some aspects, the techniques described herein relate to a computer-implemented method, wherein adjusting one or more probabilities of the action probability data includes applying a model trained to associate UI context states with adjustments to action probabilities.

In some aspects, the techniques described herein relate to a computer-implemented method, wherein adjusting one or more probabilities of the action probability data includes determining an adjustment using one or more of a rule, heuristic, or lookup table.

In some aspects, the techniques described herein relate to a computer-implemented method for context-aware neural control using a brain-computer interface, including: capturing neural signals from a user with a brain-computer interface; generating a first control output based on execution of a decoding operation on a first subset of the neural signals; transmitting, to an interactive computing device via a bidirectional human interface device (HID) communication protocol, the first control output as a standard input event of the interactive computing device; receiving, from the interactive computing device via the bidirectional HID communication protocol, user interface (UI) state metadata representing an aspect of a user interface of the interactive computing device; and generating a second control output based on execution of the decoding operation on a second subset of the neural signals and the UI state metadata.

In some aspects, the techniques described herein relate to a computer-implemented method, wherein receiving the UI state metadata includes receiving at least one of: an identifier of a currently focused UI element, an identifier of an active application, an interaction modality indicator, pointer position data, or an interaction timing parameter.

In some aspects, the techniques described herein relate to a computer-implemented method, wherein generating the second control output includes automatically selecting between discrete decoding and continuous decoding based on the UI state metadata and without explicit user-initiated mode switching.

In some aspects, the techniques described herein relate to a computer-implemented method, further including transmitting, in parallel with at least one control output, a signal quality metric representative of confidence in the decoding.

In some aspects, the techniques described herein relate to a computer-implemented method, further including generating the signal quality metric based on at least one of: a spectral power measured from a neural signal, or a confidence in a control output generated by the decoding operation.

In some aspects, the techniques described herein relate to a brain-computer interface system including: a neural interface device configured to detect a neural signal from a brain of an individual and to transmit an electronic signal representative of the neural signal; and a signal control unit including a decoder communicatively coupled to the neural interface device, the signal control unit configured to: generate a control output based on execution of a decoding operation on the electronic signal; generate a signal quality metric based on at least one of: a spectral power measured from the electronic signal, or a confidence in the control output generated by the decoder; and transmit to an interactive computing device: the control output as a standard input event of the interactive computing device; and the signal quality metric, wherein the interactive computing device is configured to present neurofeedback based on the signal quality metric.

In some aspects, the techniques described herein relate to a brain-computer interface system, wherein the signal quality metric includes a value within a range of classification confidence values.

In some aspects, the techniques described herein relate to a brain-computer interface system, wherein the signal quality metric includes a value within a range of spectral power values.

In some aspects, the techniques described herein relate to a brain-computer interface system, wherein to generate the signal quality metric, the signal control unit is configured to: determine a degree of power in one or more task-relevant neural frequency bands of the electronic signal; determine a degree of confidence in output of the decoder; and generate the signal quality metric based on the degree of power and the degree of confidence.

In some aspects, the techniques described herein relate to a brain-computer interface system, wherein the signal control unit is configured to transmit to the interactive computing device a switch identifier representing a switch to be activated.

In some aspects, the techniques described herein relate to a brain-computer interface system, wherein the signal control unit is configured to continuously evaluate and transmit the signal quality metric to the interactive computing device until the signal quality metric reaches a threshold value, and wherein the interactive computing device is configured to continuously update presentation of the neurofeedback based on receipt of the signal quality metric.

In some aspects, the techniques described herein relate to a brain-computer interface system, wherein the neurofeedback includes an animation that is continuously updated based on the signal quality metric.

In some aspects, the techniques described herein relate to a method for controlling a computing device using neural signals, including: detecting neural activity of a user with a brain-computer interface; decoding the neural activity to generate a control output; transmitting the control output to an operating system as a native input event using a human interface device protocol such that the operating system performs a user interface action; receiving, from the operating system and via the human interface device protocol, semantic user interface context information generated by the operating system, the semantic user interface context information including at least one of: (i) an identifier of a currently focused user interface element, (ii) an interaction modality associated with the focused user interface element, or (iii) an interaction timing parameter associated with an accessibility scanning or dwell process; adapting a decoding operation applied to subsequently detected neural activity based at least in part on the received semantic user interface context information, including selecting between discrete decoding and continuous decoding without user-initiated mode switching; and transmitting a subsequent control output to the operating system as a native input event using the human interface device protocol, wherein the adapting is driven by user interface state generated by the operating system rather than inferred solely from neural activity, and

In some aspects, the techniques described herein relate to a method, further including transmitting, concurrently with at least one control output, a signal quality metric representing confidence in the decoded neural activity, the signal quality metric being usable by the operating system to modify an interaction parameter of the user interface in real-time.

In some aspects, the techniques described herein relate to a brain-computer interface control system, including: a neural signal acquisition component configured to obtain neural signals representing user intent; a decoder configured to convert the neural signals into control outputs for operating a computing device; and a context input interface configured to receive user interface context information from an external operating system, wherein the decoder is configured to adapt at least one decoding parameter for converting the neural signals into the control outputs based at least in part on the received user interface context information.

In some aspects, the techniques described herein relate to a brain-computer interface control system, wherein the user interface context information includes an identifier of a currently focused user interface element.

In some aspects, the techniques described herein relate to a brain-computer interface control system, wherein the user interface context information includes an indication of whether a target user interface element supports discrete interaction or continuous interaction.

In some aspects, the techniques described herein relate to a brain-computer interface control system, wherein adapting the decoding parameter includes adjusting at least one of a classification threshold, or a timing parameter.

In some aspects, the techniques described herein relate to a brain-computer interface control system, wherein the decoder executes on a computing device separate from a computing device executing the operating system.

In some aspects, the techniques described herein relate to a brain-computer interface communication system, including: a decoder configured to generate control outputs from neural signals; and a human interface device adapter configured to transmit the control outputs to an external operating system as native input events, wherein the human interface device adapter is further configured to receive semantic user interface metadata from the operating system and to provide the semantic user interface metadata to the decoder.

In some aspects, the techniques described herein relate to a brain-computer interface communication system, wherein the semantic user interface metadata includes an identifier of a user interface element associated with a native input event.

In some aspects, the techniques described herein relate to a brain-computer interface communication system, wherein the semantic user interface metadata includes interaction timing or state information.

In some aspects, the techniques described herein relate to a brain-computer interface communication system, wherein the human interface device adapter transmits the control outputs and receives the semantic user interface metadata using a standardized human interface device protocol.

In some aspects, the techniques described herein relate to a brain-computer interface communication system, wherein the decoder is configured to modify subsequently generated control outputs based on the received semantic user interface metadata.

In some aspects, the techniques described herein relate to a brain-computer interface system, including: a neural decoder configured to generate control outputs from neural signals; a confidence evaluation module configured to generate a signal-quality metric associated with the control outputs; and a communication interface configured to transmit the control outputs and the signal-quality metric to an external operating system.

In some aspects, the techniques described herein relate to a brain-computer interface system, wherein the signal-quality metric represents confidence, strength, or reliability of the decoded neural signals.

In some aspects, the techniques described herein relate to a brain-computer interface system, wherein the signal-quality metric is transmitted concurrently with a corresponding control output.

In some aspects, the techniques described herein relate to a brain-computer interface system, wherein the signal-quality metric is continuously updated during user interaction.

In some aspects, the techniques described herein relate to a brain-computer interface system, wherein the signal-quality metric is configured to enable dynamic modification of user interaction behavior by the operating system.

In some aspects, the techniques described herein relate to a brain-computer interface control system, including: a neural signal decoder configured to generate both discrete control outputs and continuous control outputs from neural signals; and a control interface configured to transmit the discrete control outputs and the continuous control outputs to an external operating system as native input events.

In some aspects, the techniques described herein relate to a brain-computer interface control system, wherein the discrete control outputs correspond to selection or activation actions.

In some aspects, the techniques described herein relate to a brain-computer interface control system, wherein the continuous control outputs include directional and magnitude-based pointer movement.

In some aspects, the techniques described herein relate to a brain-computer interface control system, wherein selection between discrete control outputs and continuous control outputs is based on context information received from the operating system.

In some aspects, the techniques described herein relate to a brain-computer interface control system, wherein the discrete control outputs and the continuous control outputs are generated by the same decoder without requiring an explicit mode selection by a user.

In some aspects, the techniques described herein relate to a distributed brain-computer interface system, including: a neural signal decoder executing on a first computing device; a communication interface configured to transmit control outputs from the decoder to an external operating system executing on a second computing device; and a feedback interface configured to receive user interface context information from the operating system, wherein the decoder is configured to adapt generation of the control outputs based at least in part on the received user interface context information.

In some aspects, the techniques described herein relate to a distributed brain-computer interface system, wherein the first computing device is physically separate from the second computing device.

In some aspects, the techniques described herein relate to a distributed brain-computer interface system, wherein the decoder is configured to control multiple external operating systems.

In some aspects, the techniques described herein relate to a distributed brain-computer interface system, wherein the communication interface includes a wireless communication interface.

In some aspects, the techniques described herein relate to a distributed brain-computer interface system, wherein the decoder maintains state information across interactions with different operating systems.

### BRIEF DESCRIPTION OF THE DRAWINGS

Embodiments of various inventive features will now be described with reference to the following drawings. Throughout the drawings, reference numbers may be re-used to indicate correspondence between referenced elements. The drawings are provided to illustrate example embodiments described herein and are not intended to limit the scope of the disclosure. To easily identify the discussion of any particular element or act, the most significant digit(s) in a reference number typically refers to the figure number in which that element is first introduced.

FIG. 1 is a block diagram of an illustrative environment for providing context-aware decoding of neural signals according to some embodiments.

FIG. 2A is a flow diagram of an illustrative routine for decoding neural signals based on user interface context according to some embodiments.

FIG. 2B is a flow diagram of an illustrative routine for decoding neural signals based on user interface context according to some embodiments.

FIG. 3 is a flow diagram of an illustrative routine for determining the application of contributions to household contribution items associated with a household according to some embodiments.

FIG. 4 is an illustrative example user interface for providing neurofeedback according to some embodiments.

FIG. 5 is a block diagram of an illustrative signal control unit for decoding neural signals according to some embodiments.

### DETAILED DESCRIPTION

The present disclosure generally relates to decoding neural signals based in part on user interface state data received from an interactive computing device. The neural signals are decoded by a signal control unit to generate control data used to interact with the interactive computing device.

Some existing systems providing brain-computer interfaces (BCIs) have focused on decoding neural signals to control devices. However, these approaches have been limited in their ability to effectively interact with complex user interfaces. For example, some BCIs have used neural signals to provide basic control inputs, such as determining when a user intends to click a virtual button, but these systems have not been able to effectively navigate complex user interfaces (e.g., a standard user interface of a smartphone or tablet device) or perform nuanced actions (e.g., controlling a cursor that has an equivalent of two button inputs to represent a conventional two-button mouse). Other approaches have used machine learning algorithms to decode neural signals, but these algorithms have required extensive training data and have not been able to generalize well to new situations such as a dynamically changing user interface like those commonly provided to users by modern computing systems.

Some existing systems have attempted to develop brain-computer interface systems that can provide neurofeedback to users, but these systems have been limited in their ability to provide effective and informative feedback that helps the user interact with a computing system in the way the user intends. For example, some systems have provided binary visual or auditory cues to indicate whether or not a usable neural signal has been decoded, but these cues have not been able to provide detailed information about the signal quality or the user's performance. Other approaches have used more complex feedback mechanisms, such as real-time displays of brain activity, but these mechanisms can overload a user with information that is irrelevant to the user’s ability to control or interact with the computing system, paradoxically reducing the overall effectiveness of providing neurofeedback.

Therefore, such existing systems face significant limitations in reliability, accuracy, and usability, particularly when interacting with complex and dynamic user interfaces. These existing systems often decode neural signals without awareness of the current state of the user interface being controlled, leading to decoding ambiguity, increased error rates, and higher cognitive load for users. For example, traditional BCIs may rely solely on neural signal patterns to infer user intent, without incorporating contextual information about the user interface, such as the currently focused element, interaction modality, or scanning state. This lack of context-awareness results in suboptimal performance, especially in scenarios involving dynamic or multi-modal interfaces (e.g., representing a standard two-button or three-button mouse input). Furthermore, prior systems typically treat BCIs as unidirectional input devices for computing devices, failing to leverage bidirectional communication with the operating systems of the computing devices to adapt decoding behavior or provide real-time feedback to the user.

Some aspects of the BCI system of the present disclosure address some or all of the issues noted above, among others, by providing a context-aware neural decoding framework that integrates a bidirectional human interface device (HID) communication protocol for communication between the brain-computer interface and the operating system of an interactive computing device. The framework incorporates live user interface (UI) state metadata (e.g., identifiers of focused elements, control types, and interaction timing parameters) into the neural decoding process. Live UI state metadata may be UI state metadata most recently received by the BCI, or UI state metadata received by the BCI within a fixed time window ending at a current time, representing the current (or most recent) UI state. UI state metadata may be received at fixed or variable intervals. In some embodiments, UI state metadata may be received at a rate of every 200ms or less, avoiding potential desynchronization between a user’s intended actions and what is currently displayed in the UI. This contextual information may be used to dynamically adapt decoding parameters, such as classification thresholds, decoding modes, or action mappings, enabling more accurate and responsive control of the user interface for the user. The disclosed bidirectional HID communication protocol facilitates a closed-loop feedback mechanism, where the operating system provides real-time user interface context to the BCI, the BCI adapts decoding or other functionality based on the real-time user interface context, the BCI transmits both control outputs (e.g., as standard input events of the interactive computing device’s operating system) and signal quality metrics back to the operating system, and the operating system executes and updates its user interface based on the received standard input events and signal quality metrics. The BCI may dynamically adapt decoding behavior at a fixed or variable rate. For example, the BCI may determine whether to adapt decoding parameters at a time interval (e.g., 10ms, 200ms, 1s, 30s, etc.). This bidirectional interaction may allow the operating system to execute actions in response to its native input events (e.g., without translation from neural signal to action intention by the operating system) and to display or otherwise provide (e.g., through a visual indicator, an audible indicator, tactile feedback, etc.) neurofeedback indicators, adjust interaction parameters, and enhance user experience based on the reliability, consistency, or strength of the neural signals.

In some embodiments, the BCI system may receive neural signal data from a neural interface device, such as a recording device implanted in the brain or positioned to measure neural activity outside the brain (e.g., by a wearable electrode array). The BCI system may apply the neural signal data as an electronic signal for input to one or more decoders to generate control data indicating a standard input event of the interactive computing device (e.g., a switch activation or scroll), corresponding to an action intention of the user. The neural decoders may implement machine learning-based approaches, rules-based, or heuristic approaches, to decoding neural signal data generated by the recording device. The neural signal data may represent instantaneous readings, or readings generated by the recording device across a defined time window. The defined time window may be a sliding window and some portion of the neural signal data may overlap between windows. The defined time window may be a setup parameter of a decoder of the BCI system, a learned parameter of a decoder of the BCI system, user-defined (e.g., by a developer of the BCI system based on experimental or theoretical data), a user-specific time window that may or may not adapt over time with the user (e.g., a learned pattern of time window definitions for a specific user), or may be determined according to any method such that the defined time window allows the BCI system to determine user intents from neural signal data.

At least a portion of the neural decoders of the BCI system may be configured or trained to use user interface state data provided by a user device as additional information for decoding the neural signal data. User interface state data may indicate information about a user interface presented by the user device. For example, user interface state data may include the number of, identity or type of, position of, function of, or any other information of elements included in the user interface. Further, individual neural decoders may be configured for different action types. For example, a first neural decoder may be configured to determine whether a user intends to press or release a “button 1,” (e.g., decoding a discrete action) a second neural decoder may be configured to determine whether a user intends to press or release a “button 2,” and a third neural decoder may be configured to determine whether a user intends to move a mouse cursor along the display’s x and/or y axis (e.g., decoding a continuous action). In another example, a neural decoder may be configured to determine whether a user intends to perform a specific action that may be associated with a user interface such as select item, go back a screen, scroll down, scroll up, return to home screen, unlock device, and the like. A user’s intention to perform an action may be associated with a movement or other intention of the user. For example, a neural decoder configured to determine when a user intends to select a “home” button may make the determination based on analyzing neural signals to determine when the user is forming an intention to squeeze their right hand. Such a mapping between an action associated with the user interface and a movement intention is exemplary, and it should be understood that various combinations of movement or other intentions may be associated with different actions in the user interface and that such associations between actions and intensions may change based on user interface context.

Accordingly, neural decoders associated with button-type actions, also referred to as “switch” decoders, may provide a binary output indicating whether or not the user intends to select a particular user interface control or otherwise activate a particular function. In a user interface configuration that implements switches, the user interface may cycle through various options displayed in the user interface (e.g., cycle an indicator between buttons) in an order, and when a user activates the switch (e.g., intends to press the button) as represented by neural signal data associated with a particular movement intention (e.g., raising an arm, pressing down with a finger, moving a leg to the right or left, etc.) the currently-selected user interface element is “clicked” as if the user had selected the interface element with a traditional cursor or touchscreen press input.

Neural decoders associated with continuous actions (e.g., movement of a cursor along an axis) may also provide for switch-type actions. For example, a decoder may determine a user intends to move a cursor in a particular direction based on an associated movement intention (e.g., the user thinks about moving an arm left to move the cursor left on the user interface, and thinks about moving the arm right to move the cursor right on the user interface). The intention is decoded and converted into information usable by the computer presenting the user interface to move the cursor as indicated by the user’s movement intention. Additionally, the user may activate a switch-type action (e.g., representing clicking the mouse button) through a separate movement intention. Alternatively, positioning the cursor on a user interface element for a threshold time may indicate the user’s intention to cause the user interface to perform the switch-type action.

Neural decoders used by the BCI system may be determined at least in part based on the user interface state data provided to the BCI system from the user device. For example, the BCI system may determine the user is likely to intend to perform discrete actions such as button clicks where the user interface state data indicates the user interface is displaying a set of buttons, and the BCI system may determine the user is likely to perform a continuous action where the user interface is displaying a mouse cursor. The BCI system may then automatically decide to use neural decoders associated with discrete or continuous actions based on the user interface state without explicit user-initiated mode switching between the types of actions or interface control options.

The output of one or more neural decoder elements of the BCI system may be processed by a control output generator. The control output generator may determine which of the outputs of the neural decoders is most likely to represent the user’s intended action. Continuing the previous example of three neural decoders each configured to determine whether a user intends to perform an associated action, the control output generator may determine which neural decoder output is most likely to represent the user’s intended action. The control output generator may determine the most likely intended action based on a confidence value or signal strength value generated by each neural decoder and provided as part of the decoded output of the neural decoder. The control output generator may determine the most likely intended action based on the user interface state data.

The control output generator generates control data for the user device to cause the user device to execute an action within the user interface (UI) based on the user’s intention. The control data may be formatted by the control output generator according to a human interface device (HID) data object structure used by an operating system of the user device to receive input from control devices. For example, the control data may include or reference a standard input event (e.g., click, scroll, UI element activation, etc.) of the operating system. Advantageously, providing the control data according to the pre-defined HID data object structure reduces or eliminates the amount of information about the BCI system the user device needs in order to correctly map the control data to an action performable by the user device.

The user device may present a neurofeedback display to the user. The neurofeedback display may represent a neural signal strength (e.g., based on confidence in the accuracy of decoded neural data, consistency of the determined user intention over time, etc.) that can be presented to the user. The neurofeedback display may be provided to the user using various modalities (e.g., auditory, tactile, visual, etc.). For example, the neurofeedback display may be an animated bar that fills over time based on information provided to the user device by the BCI system. Advantageously, providing the neurofeedback display to the user may allow the user to determine whether their intention is being accurately captured, allowing the user to adjust their thought process if the determined intention is wrong, or to strengthen their thought process associated with the intention if the displayed intention is correct but has not yet satisfied a threshold level of signal strength or confidence. The user may therefore more accurately control the user device, reducing the number of mistakes made in controlling the user device using the BCI system, resulting in an improved user experience and reducing wasted computing resources that would otherwise have been used to correct a mistaken action.

As will be appreciated by one of skill in the art in light of the present disclosure, embodiments disclosed herein improve the ability of computing systems to support reliable, low-latency human interface interactions driven by decoded neural signals, including interactions with dynamically changing user interfaces and accessibility interaction frameworks. Moreover, the presently disclosed embodiments address technical problems inherent within computing systems; specifically, the difficulty of accurately interpreting intent-bearing neural signal data as operating-system-compatible input in the presence of rapidly changing user interface state, the difficulty of coordinating timing and action selection across bidirectional device-to-operating-system communication paths, and the difficulty of providing feedback signals that enable robust interaction control without requiring non-standard operating system integration. These technical problems are addressed by the various technical solutions described herein, including bidirectional communication in which UI state metadata is provided from an interactive computing device to a signal control unit for context-conditioned decoding, generation of standardized input events (e.g., human interface device reports) that are natively consumable by an operating system, and transmission of a signal-quality metric that enables real-time adjustment of interaction mechanics and feedback presentation based on decoded signal reliability. Thus, the present disclosure represents an improvement in computer-related technology, including operating-system input processing, user interface control, and interactive computing systems generally.

Various aspects of the disclosure will be described with regard to certain examples and embodiments, which are intended to illustrate but not limit the disclosure. Although aspects of some embodiments described in the disclosure will focus, for the purpose of illustration, on particular examples of action types, user interface elements, and the like, the examples are illustrative only and are not intended to be limiting. In some embodiments, the techniques described herein may be applied to additional or alternative types of action types, user interface elements, and the like. Additionally, any feature used in any embodiment described herein may be used in any combination with any other feature or in any other embodiment, without limitation.

### Example Neural Signal Decoding Environment

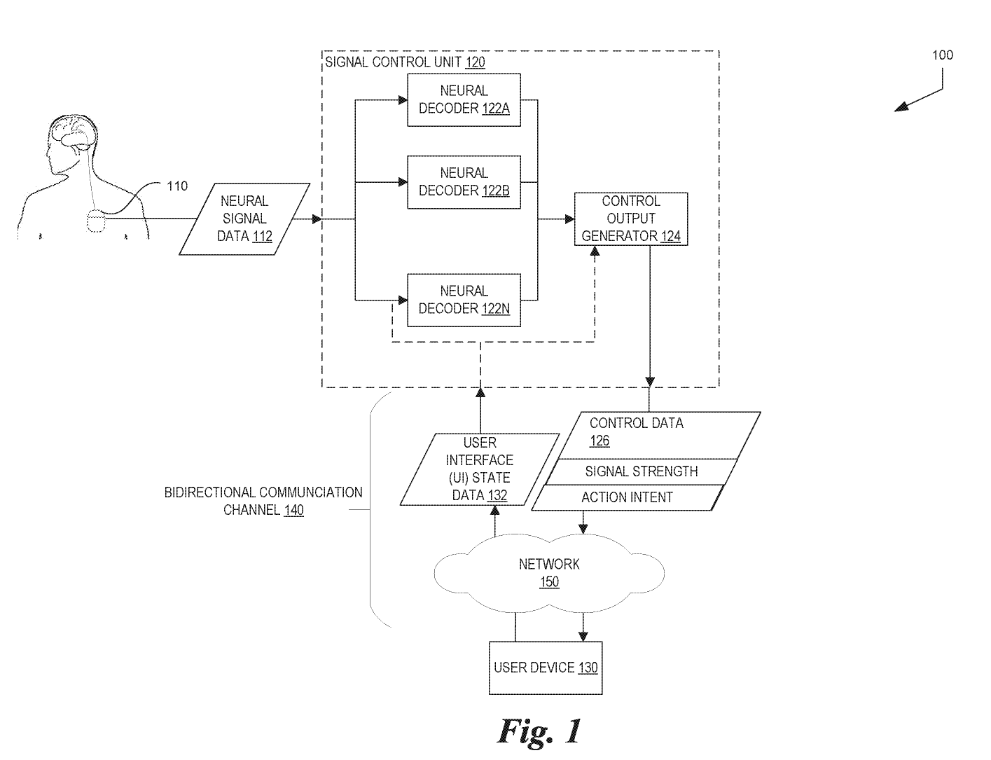

With reference to an illustrative example, FIG. 1 shows an example environment 100 for generating control data based on neural signal data and user interface state data using a context-aware signal control unit. The environment 100 includes a recording device 110 to generate neural signal data 112, a signal control unit 120 to generate control data 126, a bidirectional communication channel 140, a network 150, and a user device 130 to generate user interface state data 132 (also referred to as UI state metadata).

The recording device 110 is a device used to detect or obtain neural signal information from a brain (e.g., by a neural signal acquisition component measuring neural signals in a brain). The recording device 110 may obtain neural signal information by measuring electrical activity in a brain or portion of the brain (e.g., in a brain region associated with a function, group of neurons, individual neurons, intra-neuronal activity, etc.). For example, the recording device 110 may be a stent-electrode array configured for implantation in a body via an artery or vessel in the brain. The location and positioning (e.g., electrode depth) of the recording device 110 within the brain may be selected based on the function performed by the portion of the brain where the recording device 110 is placed. For example, the recording device 110 may be positioned at or near a motor cortex to convert action potentials in the brain associated with motor actions to neural signals that may be further processed by the signal control unit 120.

Various device configurations may be used to provide the recording device 110. Examples of such devices include those described in PCT Publication No. WO2025/0231186, titled “TRANSVASCULAR BRAIN STIMULATION,” and filed on April 30, 2025, and PCT Publication No. 2023/0240043, titled “SYSTEMS AND METHODS FOR CONTROLLING A DEVICE BASED ON DETECTION OF TRANSIENT OSCILLATORY OR PSEUDO-OSCILLATORY BURSTS,” filed on June 5, 2023, the contents of which are incorporated by reference herein in their entirety. Further, while examples discussed herein may include certain implanted or internal recording devices, in some embodiments the recording device 110 may be a non-invasive device (e.g., an electroencephalography device (EEG), functional magnetic resonance imaging scanner, a functional near-infrared spectroscopy device, etc.).

The recording device 110 may comprise a plurality of electrodes that may individually record neural activity. The individual electrodes, or combination of electrodes, may provide information about different types of intentions of the individual in whom the recording device 110 is implanted. For example, a first electrode or group of electrodes may generate neural signal information associated with the movement of a human’s index finger, and a second electrode or group of electrodes may generate neural signal information associated with movement of a human’s thumb. The recording device 110 may perform signal preprocessing operations, which may include bandpass filtering, artifact rejection, spike sorting, spectral feature extraction, temporal binning, dimensionality reduction, and normalization. The neural signal data 112 generated by the recording device 110 may be in the form of raw neural signal data that represents the measured neural activity directly, or may be neural feature data, such as a neural feature vector or sequence of neural feature vectors, representing the user’s current neural state over a window of time. The neural signal data 112 may generally represent neural activity patterns that indicate a user’s action intention, such as selecting a character displayed on a screen by the user device 130, moving a cursor, closing a switch (e.g., clicking a button), or any other user action that may be performed using the user device 130.

While an implantable stent-electrode is described above, various additional types of recording device 110 may be implemented to provide neural signal information. For example, the recording device 110 may be another type of implantable device (e.g., implanted through surgical means), or a non-implantable device (e.g., an electrode array positioned on a user’s head). In some embodiments, the recording device 110 may be a combination of different types of recording devices 110 that may provide different types of information (e.g., a first recording device 110 providing information about the squeezing of individual fingers and a second wearable recording device 110 providing information about gross hand motions), or provide the same type of information (e.g., an implantable recording device 110 and a wearable recording device 110 measuring the same neural information). The recording device 110 may convert any recorded information into a standard neural signal data 112 output for processing by the signal control unit 120.

The signal control unit 120 is a computing device that may implement various models to generate control data 126. The signal control unit 120 includes one or more neural decoders 122A, 122B, …, 122N (collectively 122A – 122N), and a control output generator 124.

The one or more neural decoders 122A – 122N receive neural signal data 112 from the recording device 110 and execute decoding operations to provide decoded neural signal information to the control output generator 124. The one or more neural decoders 122A – 122N may be implemented as a multi-layer perceptron with a softmax output layer over a set of discrete output classes, a recurrent neural network such as a long short-term memory network or gated recurrent unit, a transformer decoder, a temporal convolutional network, or any combination of the preceding options. Individual neural decoders 122 may receive neural signal data 112 from distinct portions of the recording device 110. For example, a first neural decoder that is configured to map an action to an intention to move an index finger may receive neural signal data 112 from a portion of the recording device 110 that captures neural information associated with movement of the index finger in the motor cortex. In this example, a second neural decoder 122 that is configured to map a second action to an intention to move a hand may receive neural signal data 112 generated by a portion of the recording device 110 that captures neural information associated with gross hand movement. In some embodiments, the one or more neural decoders 122A - 122N may be configured to receive neural signal data 112 generally and classify the neural signal data as one of a plurality of actions, where individual decoders may not be specifically trained to map individual actions directly to individual associated intentions. Instead, a neural decoder 122 in this configuration may generate an output indicating a most likely intention from among a set of potential intentions available to that neural decoder 122. For example, a neural decoder 122 may receive the neural signal data 112 and generate a classification output indicating that the user intends to press a “button 1” from among a plurality of available buttons that could be pressed.

The one or more neural decoders 122A - 122N may further be configured to process user interface state data 132 when determining an intention associated with neural signal data 112. The user interface state data 132 may be received from a user device 130 and may indicate a current state of the user interface presented by the user device 130. The user interface state data 132 may include a semantic description of user interface context information. The user interface state data 132 may include a timestamp. The neural decoder 122 may use the timestamp in the user interface state data 132 to align the user interface state data 132 with neural signal data 112 based on a timestamp included with the neural signal data 112 received from the recording device 110. Additionally, the user interface state data 132 may include a spatial arrangement of elements, an indication of a currently highlighted or focused element, an indication of whether a target user interface element (e.g., a currently selected element or input method) supports discrete or continuous interaction, a history of recent actions, previous patterns of selections by the user, a current time of day, a time since a last action was performed by the user device 130 based on control data 126, a location of the user device 130, ambient conditions in an area around the user being monitored by the recording device 110 (e.g., lighting, temperature, etc.), eye-tracking data, or other sensor inputs received by the user device 130.

To perform a decoding operation, a neural decoder 122 may implement a machine learning model that has been trained to interpret neural signal data 112 and/or user interface state data 132 to determine an action intention. The machine learning model of an individual neural decoder 122, also referred to herein as a decoder model, may be trained for a specific type of action intention. For example, a first neural decoder 122 may be trained to determine whether neural signal data 112 indicates a user intends to press a first button, a second neural decoder 122 may be trained to determine whether neural signal data 112 indicates a user intends to press a second button, and a third neural decoder 122 may be trained to determine a direction a user intends to move a cursor in two or three dimensions and/or generate a magnitude-based indication of the motion of the cursor.

Where a neural decoder 122 implements a machine learning model, the signal control unit 120, or the neural decoder 122, may generate neural feature data as an embedding (e.g., as a neural feature vector) representing at least a portion of the neural signal data 112 for input to the neural decoder 122. The signal control unit 120 or neural decoder 122 may further generate UI state feature data as an embedding (e.g., as a context feature vector) based on the user interface state data 132 for input to the neural decoder 122. To generate an embedding, the neural decoder 122, or an encoder provided by the signal control unit 120, may transform data (e.g., the neural signal data 112 or the current user interface state data 132) into a dense embedding vector. An embedding generated from the current user interface data 132 may be referred to as a context embedding vector (or simply a “context embedding” for brevity); an embedding generated from neural signal data 112 may be referred to as a neural embedding vector (or simply a “neural embedding” for brevity). The signal control unit 120 may generate an embedding based on use of a lookup table, where a state identifier associated with the neural signal data 112 or user interface state data 132 allows the signal control unit 120 to identify a previously-generated embedding for that information. The embedding may be generated by a neural network or transformer-based encoder trained to operate on a tokenized version of the neural signal data 112, user interface state data 132, or combination of the two.

A context feature vector (which may be a context embedding) may be concatenated with a neural feature vector (which may be a neural embedding) to generate decoder input prior to a unified decoding stage. The combined embedding representation, or individual embeddings generated from the neural signal data 112 or user interface state data 132, may be generated to have a size (e.g., a dimensionality of a vector) that will correspond to or otherwise not exceed an input size limit of a machine learning model of the neural decoder 122. The neural decoder 122 may then analyze the embeddings to determine the user intention associated with the neural signal data 112. In this way, the neural decoder 122 jointly conditions its output on both the neural signal data 112 and user interface state data 132.

A neural decoder 122 may implement a rules-based approach or a heuristic approach to determining an action intention from the received neural signal data 112. For example, activity information in different neural frequency bands may be associated with an intention. When the neural decoder 122 determines the pattern of neural activity in the neural signal data 112 matches an expected pattern of neural activity for an intention, the neural decoder 122 may determine that the neural signal data 112 indicates the intention. In some embodiments, the same neural decoder 122 may implement both a rules-based or heuristic approach and a machine learning model approach to decoding neural signal data 112. In some embodiments, a chain of neural decoders 122 may be used where a first neural decoder 122 in the chain uses a rules-based or heuristic approach and a second neural decoder 122 implements a machine learning-based approach.

The one or more neural decoders 122A - 122N may further generate action probability data, such as a confidence value or other value that represents a determined probability for an action associated with the neural decoder 122. For example, multiple neural decoders 122, or the same neural decoder 122, may be trained to determine whether a user intends to enact any one of a plurality of candidate actions. The neural decoder 122 associated with an action may then generate probability data indicating a confidence that the neural signal data 112 indicates the user intends to perform the associated action. In some embodiments, the probability or confidence may be represented as a signal strength by the control output generator 124 as described below.

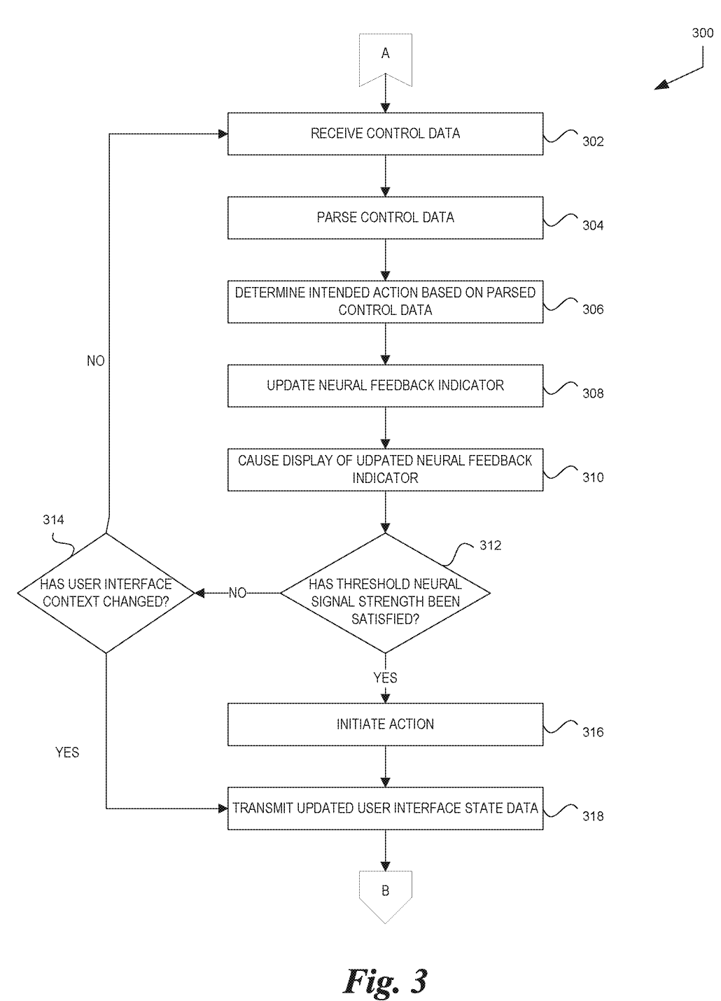

The control output generator 124 is configured to generate control data 126 based on the decoded neural signal data 112 generated by the one or more neural decoders 122A - 122N. The control data 126 may be generated based on a subset of the decoded neural signal data 112 generated by the one or more neural decoders 122A - 122N. The control output may be a standardized data object, such as a HID event object, that is interpretable by the operating system of the user device 130. The control data 126 may include an indication of signal quality, or confidence in the action indicated by the control data 126, for use or display by the user device 130 as described in further detail with respect to FIG. 3 below herein as part of a neurofeedback display.

The network 150 may be a publicly-accessible network of linked networks, some or all of which may be operated by various distinct parties, for example the Internet. In some cases, network 150 may include a private network, personal area network, local area network, wide area network, cellular data network, satellite network, etc., or some combination thereof, some or all of which may or may not have access to and/or from the Internet. In some cases the network 150 may be a Bluetooth, or other local wireless connection. The network 150 may be a direct wired or wireless connection between the signal control unit 120 and the user device 130. The bidirectional communication channel 140 may be implemented via the network 150 to enable communication of user interface state data 132 from the user device 130 to the signal control unit 120 and control data 126 from the signal control unit 120 to the user device 130.

The user device 130 is any device a user may want to control using a signal control unit 120. For example, a user device 130 may be a smartphone, laptop, tablet, or other computing device capable of receiving control data 126 from the signal control unit 120 to execute an action in a user interface associated with an intention represented in the control data 126.

### Example Signal Decoding Routines

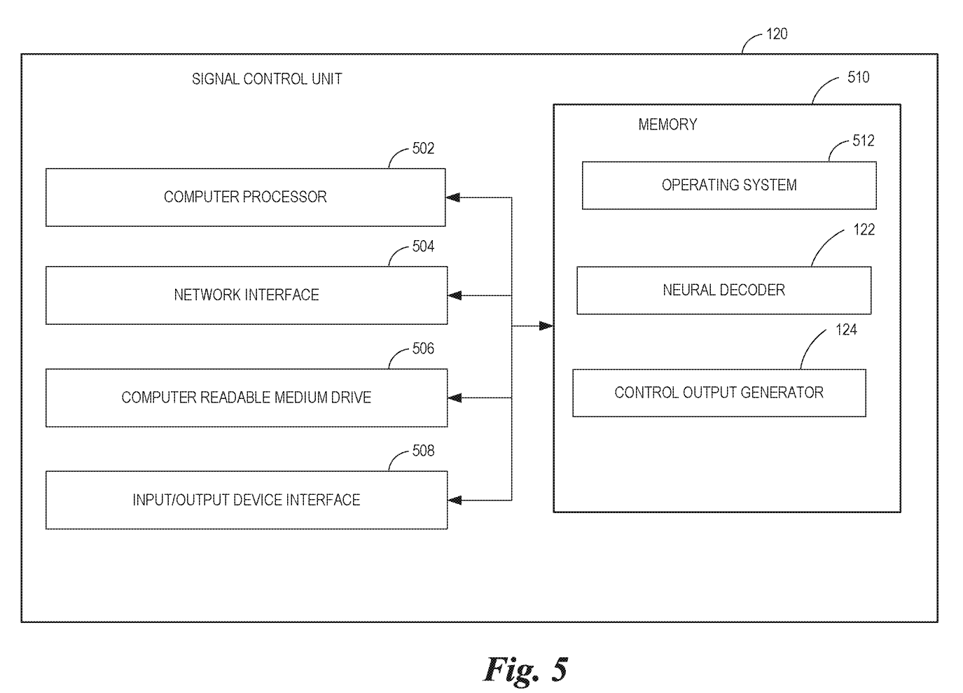

When a routine described herein (e.g., routine 200, 250, or 300) is initiated, a set of executable program instructions stored on one or more non-transitory computer-readable media (e.g., hard drive, flash memory, removable media, etc.) may be loaded into memory (e.g., random access memory or RAM) of a computing device, such as the signal control unit 120 shown in FIG. 5, and executed (e.g., a computer-implemented method) by one or more processors. In some embodiments, the routines 200, 250, or 300, or portions thereof, may be implemented on multiple processors, serially or in parallel.

Further, routines described herein may refer, in part, to an order of operations, blocks, or steps. It should be understood that any reference to an order of the blocks of a routine is intended to aid in the explanation of the routine only, and does not imply that such an order is required for performing the operation described by the routine. Blocks of the routines described herein, therefore, should be understood to be capable of being performed before, after, and/or simultaneously with any other block of the routine. Additionally, various blocks of routines described herein may be performed repeatedly even where no loop is explicitly shown. Such loops may be excluded from the description of a routine for the purpose of clarity, and does not require that a block be performed only once before the routine proceeds. Further, some blocks in a routine described herein may be omitted or rearranged such that a routine (e.g., routines 200, 250, or 300), begins at a different block from the block show in the figure.

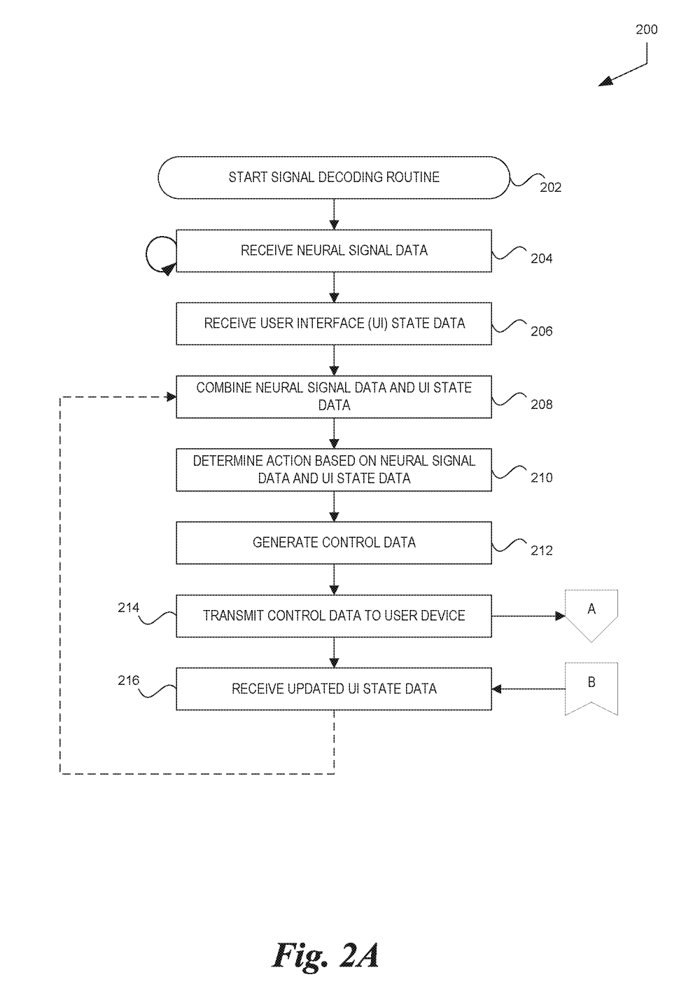

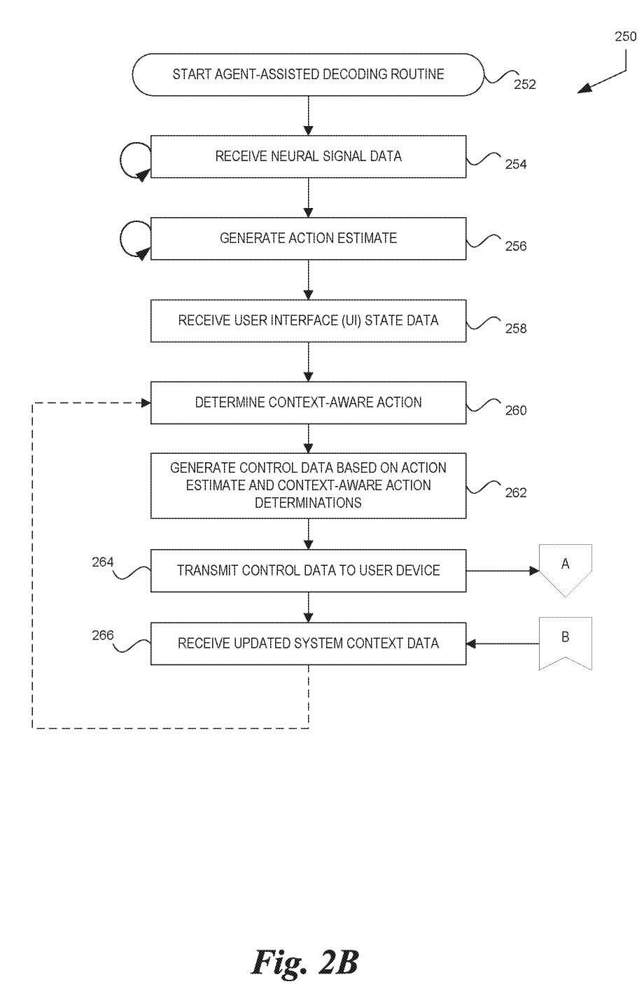

FIG. 2A illustrates example routine 200 for decoding neural signal data based on a user interface context. In this example routine 200, the one or more neural decoders 122A – 122N are context-aware neural decoders that are trained to generate decoded neural signal data based on a combination of the neural signal data 112 and the user interface state data 132.

The routine 200 begins at block 202, for example in response to a user initiating a connection between a signal control unit 120 and a user device 130 (e.g., via the network 150), or in response to a user device 130 confirming a connection between the signal control unit 120 and the user device 130 (e.g., by the signal control unit receiving an acknowledgment or user interface state data 132 from the user device 130). Beginning the routine 200 may include initializing at least one neural decoder 122 to begin decoding neural signal data 112 received from the recording device 110.

At block 204, the signal control unit 120 receives neural signal data 112 from the recording device 110. The signal control unit 120 may continuously receive subsequently detected or updated neural signal data 112 whether or not corresponding updated user interface state data 132 is received. Neural signal data 112 may include information related to neural oscillations of a user, including changes in neural oscillations, over time. Neural oscillations may be grouped into bands that are defined by frequency ranges. For example, neural signal data 112 may include information related to an alpha band of about 7 Hz to 12 Hz, a theta band of about 4 Hz to 7 Hz, a gamma band of about 70 Hz to 135 Hz, a delta band of about 0.1 Hz to 3 Hz, a mu band of about 7.5 Hz to 12.5 Hz, a sensorimotor rhythm band of about 12.5 Hz to 15.5 Hz, or any other band defining a range of frequencies of neural oscillations. The neural signal data 112 may be received by the signal control unit 120 as a data object structuring information about one or more of the described frequency bands. The neural signal data 112 may be received as individual data objects associated with each frequency band for which the recording device 110 generates measurement values.

At block 206, the signal control unit 120 receives user interface state data 132 from the user device 130 regarding one or more aspects of the UI of the user device 130. The user interface state data 132 may include metadata representing a current status of the UI (e.g., on or off, locked or unlocked, rendering or idle), an interaction modality indicator (e.g., an indicator of whether the UI is operating in scanning mode for switch input, or is displaying a cursor), current UI configuration (e.g., position or type of interactable elements displayed in the user interface), current active application(s) displayed on the UI, current UI element focus, current (or most recent) interaction associated with the UI, UI interaction history (e.g., a set of actions previously performed with respect to the user interface displayed by the user device 130), a timestamp indicating a time at which the user device 130 generated the user interface state data 132, or any other information associated with the current user interface presented by the user device 130. For example, the user interface state data 132 may include an identifier or descriptor for an element of the user interface that has a current focus. The user interface state data 132 may include further information about the element with the current focus, for example an application that causes presentation of the element, a type of interaction available (e.g., a single click interaction, different click interactions for various button types, etc.), a location of the element within the user interface, a size of the element, an effect of selecting the element (e.g., typing a letter for a virtual keyboard, exiting an application, saving a current progress in a document, scrolling a page, deleting text, pasting copied text, etc.), or any other information available to the user device 130 about the element. The user interface state data 132 may include metadata indicating a current dwell time of a selection or cursor on an element of the user interface, a remaining dwell time until an action is registered, a total dwell time before an action is registered, or other information related to the selection of an element and the activation of the element based on the selection.

The user interface state data 132 may be received by the signal control unit 120 in the form of a scan information report generated by the user device 130. The scan information report may include information about a number of interactable elements displayed by the user device 130, or a type of control (e.g., switch control, scanning, etc.). The user device 130 may omit some information from the scan report. For example, the user device 130 may omit information indicating a key of a keyboard that is currently selected to preserve user privacy. Examples of information included in the scan information report may include an identifier (e.g., a numerical value associated with) a selected interactable element, a total number of interactable elements displayed on a display of the user device 130, a scan cycle value that may be used by the signal control unit 120 to distinguish between different scan passes or groups, an element type of a currently focused interactable element or group of elements, and a scanning latency indicating a time between when the user device 130 receives an instruction from the signal control unit 120 and when the action associated with the instruction is executed.

At block 208, the signal control unit 120 combines the neural signal data 112 and the user interface state data 132 to generate a combined input data object for the at least one neural decoder 122. Advantageously, generating the combined input data object for the neural decoder 122 may allow the neural decoder 122 to be inherently context-aware as the learned parameters of a model implemented by the neural decoder 122 are based on correlations between current interface states represented in the user interface state data 132 and the neural signal data 112 with specific intentions. Combining the neural signal data 112 and the user interface state data 132 may be based on a timestamp associated with the neural signal data 112 and a timestamp associated with the user interface state data 132 to maintain temporal alignment between information received at the signal control unit 120. The combined data may be converted by the signal control unit 120 into a feature vector including at least a portion of the neural signal data 112 and the user interface state data 132. For example, an encoder may be used by the signal control unit 120 to generate an embedding representation of the neural signal data 112 and the user interface state data 132 for input into the neural decoder 122 as described above. In some embodiments, the neural signal data 112 may be converted into a first embedding, and the user interface state data 132 may be converted into a second embedding, and the first and second embeddings may be concatenated prior to input to a neural decoder 122.

In some embodiments, a context embedding model may be trained using an autoencoder architecture to learn compact, meaningful vector representations of user interface data in an unsupervised or self-supervised manner. An autoencoder is a neural network trained to reconstruct its input. It consists of two main parts: an encoder that maps the input data to a lower-dimensional vector (the embedding or latent representation), and a decoder (separate from the neural decoder 122) that reconstructs the original input from that embedding. The embeddings are learned by forcing the autoencoder to compress information while still retaining what is necessary for reconstruction. In some embodiments, an encoder-decoder does not necessarily reconstruct its original input, but rather generates other related output. For example, an encoder-decoder may be trained to predict the visual state of a user interface based on user interface state data 132. In either case, the encoder portion of the neural network encodes the user interface state data 132 and passes the encoded user interface state data 132 to the decoder, which reconstructs the input or produces related output such as a prediction of a current user interface that corresponds to the user interface state data 132. After training, the encoder portion of the trained autoencoder (or other encoder-decoder architecture) may be deployed as a context embedding model that generates context embeddings as encoded representations of user interface state data 132. Similarly, a neural embedding model may be trained using an autoencoder architecture or other encoder-decoder architecture (separately from the neural decoder 122). The encoder portion of this autoencoder may be deployed as the neural embedding model that generates neural embeddings as encoded representations of neural signal data 112.

Where the signal control unit 120 includes a plurality of neural decoders 122, the signal control unit 120 may generate multiple distinct combined input data objects for the neural decoders 122 (e.g., embeddings generated by different encoders or other embedding models, or embeddings including different portions of the neural signal data 112 and user interface state data 132) that may include portions of the neural signal data 112 and user interface state data 132 associated with the respective neural decoder outputs. For example, a first neural decoder 122A may be trained to generate a single button output (e.g., a binary value representing the button state), and a second neural decoder 122B may be trained to generate a movement for a 2-dimensional (2D) cursor. The signal control unit 120 may provide a first combined input data object to the first neural decoder 122A including user interface state data 132 useful for determining whether a button that is clickable is currently selected in the user interface of the user device 130. In this example, the signal control unit 120 may provide a second combined input data object to the second neural decoder 122B including user interface state data 132 useful for determining a current position of a 2D cursor in the user interface, elements in the user interface that may be selectable by the 2D cursor, a dwell time of the 2D cursor on a selectable object, or any other information useful for determining whether to move the 2D cursor and in what direction a user may intend to move the 2D cursor.

At block 210, the control output generator 124 determines an action based on the output generated by the at least one neural decoder 122. The control output generator 124 may determine an action directly from the output of the neural decoder 122. The output of the neural decoder 122 may represent a discrete selection from among currently visible candidates on the user interface presented by the user device 130, a continuous control signal (e.g., a magnitude and/or direction of movement for a cursor on a display), a high-level command (e.g., a command associated with a particular switch and its associated movement intention), or a probability distribution over a range of potential control options (e.g., a vector or other representation of probabilities associated with respective potential actions the user could take based on the user interface state data 132). For example, the control output generator 124 may receive an output from one neural decoder indicating a user intends to virtually press a button (e.g., represented as selecting a currently highlighted object in the user interface) and may generate control data 126 to represent the user’s intention to virtually press the button as described below herein. The control output generator 124 may determine the action based at least in part on a confidence value received from the at least one neural decoder 122. For example, where one neural decoder 122 is implemented by the signal control unit 120, the control output generator 124 may determine whether or not an action is intended by the user based on a confidence value included with the output of the neural decoder (e.g., by comparing the confidence value to a threshold value).

Where two or more neural decoders 122 are implemented by the signal control unit 120, the control output generator 124 may select from among a plurality of actions associated with different neural decoders 122 based at least in part on the confidence value provided by each neural decoder 122 to the control output generator 124. For example, the control output generator 124 may compare the confidence values of each output received from a neural decoder 122 and use the output associated with the highest confidence value. In another example, where the user interface state data 132 indicates an action type associated with a particular neural decoder 122 is not available, the associated neural decoder 122 may provide a confidence value or other indication that the output of that neural decoder 122 should not be selected (e.g., by setting the confidence value to a minimum value). Where multiple neural decoders 122A – 122N are implemented, the control output generator 124 may further determine whether any output is associated with a confidence value that satisfies a threshold value. Additionally, where multiple neural decoders 122A – 122N are implemented, the signal control unit 120 may select one or more of the available decoders 122 based at least in part on the user interface state data 132. For example, where the user interface state data 132 indicates the user interface of the user device 130 is operating in a scanning mode, the signal control unit 120 may use a neural decoder 122 that is associated with determining when a user intends to select a currently highlighted item by the scanner, and may choose not to use, or to ignore the output of, a neural decoder 122 that is associated with moving a cursor in two dimensions.

Where the control output generator 124 determines that no confidence value satisfies the threshold value, the control output generator 124 may generate control data 126 indicating no action, or wait to generate control data 126 until decoded neural signal data received from a neural decoder 122 satisfies the threshold value.

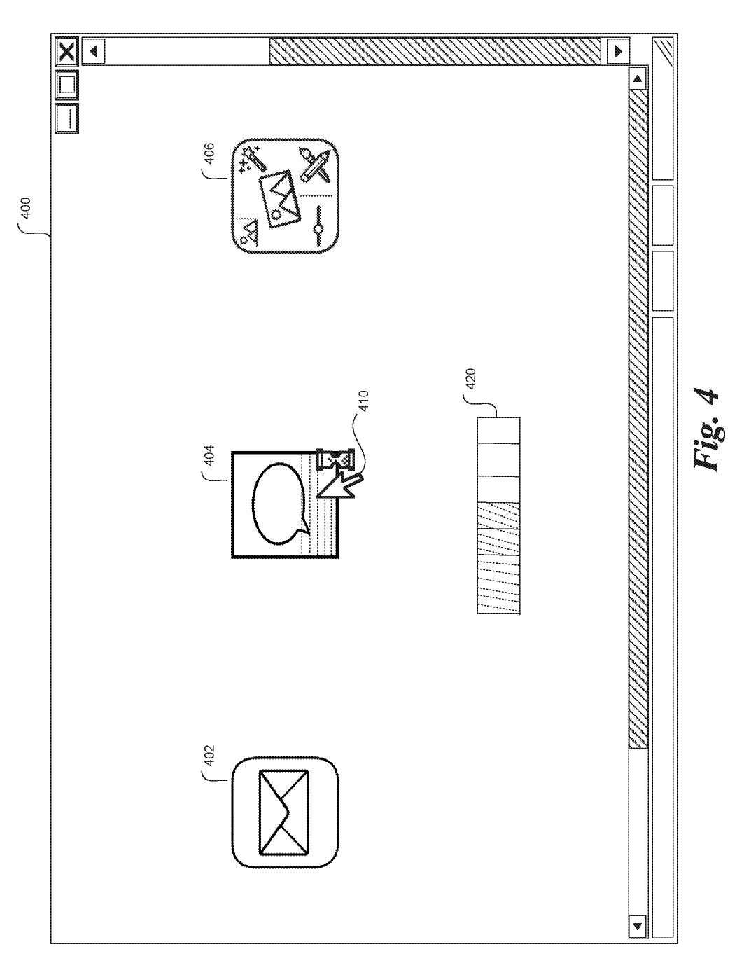

At block 212, the control output generator 124 generates control data 126 for the user device 130. Control data generated by the control output generator 124 may include an indication of an action. In some embodiments, control data generated by the control output generator 124 may also include an indication of a confidence value associated with the action, a signal strength, or another signal quality metric. For example, the confidence value may be a confidence value generated by a neural decoder 122. The signal strength or confidence may be determined based on a spectral power value of a portion of neural signal data associated with the output of a neural decoder 122, a distance from a decision boundary of the output of the neural decoder 122, a degree of power of a portion of neural signal data, or a combination of these values. The signal strength may represent a current measure of neural-control reliability, intent salience, or output confidence. The signal strength may be determined for task-relevant neural frequency bands based on, for example, a user interface state or input control method being used. The signal strength may not be derived from the same embedding representation used to decode the neural signal data 112, or may use a separate neural decoder 122 from the neural decoder 122 that generated the output used by the control output generator 124 to determine the user’s intended action. For example, a neural decoder 122 may determine the signal strength based on raw neural recordings, intermediate signal features (e.g., bandpower, spike rate, signal-to-noise ratio (SNR), artifact burden), decoder outputs (e.g., posterior probability, decision margin), temporal stability across cycles, or other quality estimators (including estimators produced by modules separate from the decoder), or a combination of any of the preceding. In some embodiments, the signal strength may be transmitted by the signal control unit 120 as a distinct output from the action information. For example, the signal control unit 120 may continuously provide signal strength information (e.g., quantified as a signal quality metric) to the user device 130 even when an action is not provided in the control data 126 to the user device 130, and may allow the user device 130 to continuously render a current neurofeedback display (e.g., as discussed with respect to FIG. 4 below herein).

In some embodiments, the neural decoder 122 may generate the confidence or signal strength value. In some embodiments, a confidence value generated by a neural decoder 122 may be based at least in part on the spectral power of a portion of the neural signal data.

The terms confidence value, signal strength value, and signal quality metric may be used interchangeably herein to represent the likelihood that an output of a neural decoder 122 or the control output generator 124 has accurately determined a user’s intended action from the neural signal data 112.

The control output generator 124 may generate the control data 126 in the form of a human interface device (HID) report according to a HID protocol used by an operating system of the user device 130. For example, the operating system of the user device 130 may include in its HID protocol an object type representing a single finger touch on a touchscreen, a left mouse click, or other input type associated with selecting a displayed element. The control output generator 124 may then format the control data 126 according to the HID protocol definition for such an action. The control output generator 124 may store HID protocol definitions in the signal control unit 120. The control output generator 124 may request HID protocol definitions from the user device 130.

The control output generator 124 may generate various types of HID reports according to a HID protocol. For example, the control output generator 124 may generate a BCI input signal report indicating a signal quality as determined by the signal control unit 120 of a neural signal. The signal quality report may include an indication of a button or action associated with the neural signal being evaluated, and an indication of a neural activity strength. The neural activity strength may be provided as a scaled or normalized value between a lower and upper bound (e.g., a value bounded by 0 and 255, inclusive of the bounds, represented by a single byte).

In another example, the control output generator 124 may generate a BCI input button report. The input button report may include an input value for a set number of buttons indicating whether any of the buttons is in an active state. The report may include the current state (e.g., inactive or active) for each button, or may indicate a change in state of a button. Each button may be associated with an action defined by the signal control unit 120 or the user device 130. Examples of actions include, but are not limited to, select, move to next item, move to previous item, toggle menu, activate, start sequential navigation (e.g., scan mode), stop sequential navigation, trigger automation (e.g., a predefined set of actions associated with a single button press), toggle application switcher view, home, toggle notification view, assistant, volume up, volume down, toggle dictation, toggle an accessibility feature, toggle quick settings view, escape, scroll up, scroll down, scroll left, or scroll right.

In a further example, the control output generator 124 may generate a BCI input pointer report. The input pointer report may be associated with movement of an on-screen cursor by the external operating system of the user device 130. The input pointer report may include values associated with x, y, and/or z axis movement. The values associated with the x, y, and/or z axis movement may be signed values, where the sign is used to indicate a direction of movement along an associated axis. The values in the input pointer report associated with each axis may be relative values, indicating a change in position relative to a previous position, or absolute values, indicating an absolute position (e.g., as a pixel location) on a display of the user device 130.

In an additional example, the control output generator 124 may generate a BCI input item selection report. The set of input items available for selection may be determined by the signal control unit 120 or a neural decoder 122 based on the user interface state data 132 received from the user device 130. The user interface state data 132 may associate different interactable elements (e.g., a button, scroll bar, etc.) with an identifier value. The user interface state data 132 received by the signal control unit 120 may include, for each interactable element, an indication of a type of interactable element (e.g., a button type) and the identifier value associated with the interactable element. Interactable elements may include elements not currently displayed by a user interface of the user device 130. The control output generator 124 may generate the input item selection report indicating the interactable element that has been selected by including the identifier associated with the interactable element.

At block 214, the control output generator 124 transmits the generated control data 126 to the user device 130. The control data 126 may be provided by the control output generator 124 following a time delay. For example, based on timestamp information included in the neural signal data 112, and timestamp information included in the user interface state data 132, the control output generator 124 may adapt the latency in providing the control data 126 to the user device 130 to provide a consistent delay between a user’s intention represented in the neural signal data 112 and a change in the user interface based on the user’s intention, thereby providing a more consistent user experience and allowing the user to better plan further actions. The control output generator may provide updated control data 126 to the user device 130 within a target time range or at fixed intervals. For example, the control output generator 124 may provide control data 126 to the user device 130 at a defined timing interval (e.g., 5ms, 25ms, 200ms, 500ms, etc.). The control output generator 124 may delay or otherwise modify the transmission of the control data 126 to provide a more consistent or predictable user experience based on the timestamp information described above herein.

At block 216, the signal control unit 120 receives updated user interface state data 132 from the user device 130. The user interface state data 132 may indicate any changes (e.g., a delta report showing relative changes) in the user interface between when user interface state data 132 was previously received by the signal control unit 120 and a time when the user interface state data 132 was sent by the user device 130 to the signal control unit 120. The user interface state data 132 may indicate a current state of the user interface provided by the user device 130 that may or may not reference a previous state of the user interface. The updated user interface state data 132 may include a timestamp generated by the user device 130 indicating when the updated user interface state data 132 was generated or transmitted to the signal control unit 120. The routine 200 may continue to execute based on updates to the user interface state data 132 and updated neural signal data 112 received from the recording device 110 and may generate subsequent control data 126.

FIG. 2B illustrates example routine 250 for decoding neural signal data and determining a context-aware action based on a user interface context and the decoded neural signal data, using an agent-assisted determination of a user’s intended action according to some embodiments. In contrast to the example routine 200 described above, in the routine 250 at least one neural decoder 122 of the one or more neural decoders 122A – 122N is trained to generate decoded neural data based on neural signal data 112 without user interface state data 132. Optionally, at least one neural decoder 122 of the one or more neural decoders 122A - 122N may be trained to generate decoded neural data based on a combination of neural signal data 112 and user interface state data 132. An agent of the signal control unit 120 (e.g., implemented in the control output generator 124) may then combine the decoded neural signal data 112 generated by a neural decoder 122 with the user interface state data 132, or an output generated by a neural decoder 122 based on a combination of the neural signal data 112 and user interface state data 132, to determine the user’s intended action when generating control data 126. The routine 250 begins at block 252, for example as described above with respect to block 202 of FIG. 2A.

At block 254, the signal control unit 120 receives neural signal data 112 from the recording device 110 as described above with respect to block 204. As described above, the signal control unit 120 may continuously receive updated neural signal data 112 over time regardless of whether corresponding updated user interface state data 132 is received.

At block 256, one or more neural decoders 122A – 122N generate an action estimate based on the neural signal data 112. The neural signal data 112 may be provided to a neural decoder 122 as an embedding (e.g., a feature vector) representation generated by an encoder. The neural signal data 112 may be provided directly to the neural decoder 122, and an encoder or other embedding model of the neural decoder 122 may generate an embedding for that neural decoder 122. The neural decoder 122 may generate an action estimate based on the neural signal data 112 and an action type associated with the neural decoder 122. For example, a neural decoder 122 associated with a click action may generate an action estimate indicating whether or not the neural signal data 112 represents a user intention to click or release a click. In another example, a neural decoder 122 associated with a scroll action may determine whether the neural signal data 112 indicates a user’s intent to scroll, an axis along which the user intends to scroll, a direction along the axis in which the user intends to scroll, and/or a user’s intent to stop scrolling. The neural decoder 122 used to generate the action estimate may differ from a neural decoder 122 used to determine an action based on both neural signal data 112 and user interface state data 132, such as the neural decoder(s) 122 described with respect to routine 200. For example, a neural decoder 122 used in routine 250 to generate the action estimate may be trained on training data including neural signal data 112 but not training data including user interface state data 132. The action estimate may include a confidence value or signal strength information indicating a confidence of the neural decoder 122 that the action determination is correct, or a signal strength as determined from the neural signal data 112 or included in the neural signal data 112.

Similarly to the receipt of updated neural signal data 112, the one or more neural decoders 122A – 122N may generate updated action estimates based on updated neural signal data 112 received from the recording device 110 regardless of whether corresponding updated user interface state data 132 is received by the signal control unit 120 from the user device 130. Where multiple neural decoders 122A – 122N are implemented, the signal control unit 120 may select one or more of the available decoders 122 based at least in part on the user interface state data 132. For example, where the user interface state data 132 indicates the user interface of the user device 130 is operating in a scanning mode, the signal control unit 120 may use a neural decoder 122 that is associated with determining when a user intends to select a currently highlighted item by the scanner, and may choose not to use, or to ignore the output of, a neural decoder 122 that is associated with moving a cursor in two dimensions. The selected neural decoder 122 may be selected by an agent implemented by the control output generator 124, such as an agent described below.

At block 258, the signal control unit 120 receives user interface state data 132 from the user device 130 as described above with respect to block 206. In some embodiments, the routine 250 may begin at block 258 (e.g., block 258, alone or along with block 260, may precede block 254). As discussed previously, the order of blocks within the routine 250 is exemplary and the order of blocks may be changed.

At block 260, the signal control unit 120 determines a context-aware action. The determination of the context-aware action may be performed similarly to the context-aware decoding described above with respect to block 210. For example, the signal control unit 120 may concatenate an embedding generated based on the neural signal data 112 and an embedding generated based on the user interface state data 132. Portions of the neural signal data 112 or user interface state data 132 may be excluded from the information provided to a neural decoder 122, for example, based on the action associated with the neural decoder 122.

In some embodiments, the context-aware action determination may be made by the control output generator 124 based on the output of a neural decoder 122, where the output of the neural decoder 122 was generated based on neural signal data 112 without user interface state data 132. In such embodiments, the control output generator 124 may receive the user interface state data 132 and implement an agent trained to determine the context-aware action. The agent may interpret the decoded neural signal data provided by the neural decoder 122 in the context of the user interface state data 132 to generate a decoded output that the control output generator 124 uses to generate control data 126. For example, if the user interface state data 132 indicates that a button for closing a window in the user interface is highlighted, the control output generator 124 may lower the confidence or weight associated with an output of a neural decoder 122 that generates action information for scrolling and increase a confidence or weight associated with an output of a different neural decoder 122 that generates action information for performing a click action.

In such embodiments, the agent may compare the output of the neural decoder 122 to the user interface state data 132 and apply a weight to one of a plurality of available actions (e.g., to increase or decrease the probability of an action or a confidence in the action), or otherwise interpret the decoded neural signal data provided by the neural decoder 122 in the context of the user interface state data 132 to generate control data for the intended action in a context-aware manner. Further, the control output generator 124 may identify relevant feature information from an embedding representation of the user interface state data 132 based in part on the decoded output of the neural decoder 122, or identify relevant feature information from the decoded output of the neural decoder 122 based on the user interface state data 132. In either implementation, or where both are implemented, the control output generator 124 may reweight probabilities or confidence values associated with particular actions available to the user in the user interface to more accurately determine the user’s intended action than would be possible without use of the user interface state data 132.

The agent implemented by the control output generator 124 may be trained to associate interface context states represented in the user interface state data 132 with adjustments to intention probabilities or confidences in the decoded output of a neural decoder 122. Pretraining approaches to train the agent may include reinforcement learning, in which the agent learns a policy that maximizes a reward signal tied to successful task outcomes (e.g., correct selection rate, information transfer rate, or task completion speed) in a simulated or recorded interaction environment; supervised learning on historical interaction data in which ground-truth user intentions are known and the agent learns to upweight actions that match the true intention given the observed context, and/or downweight actions that do not match the true intention based on the observed context; imitation learning in which the agent learns from expert demonstrations of optimal action weighting given context; or any combination thereof. In some embodiments, a plurality of agents may be implemented by the control output generator 124, and the output of the various agents may be reweighted based on comparing the outputs to each other or the user interface state data 132.

In some embodiments, the agent implemented by the control output generator 124 may be a rules-based agent that uses a set of defined rules, heuristics, or lookup tables that encode domain knowledge about the interface and its expected interaction patterns. For example, the agent may refer to rules that suppress selection-type actions when the interface is in a scrolling mode, that increase the probability of a confirmation action when a dialog box is in focus, that mask actions corresponding to interface elements that are not currently visible or interactable, or that adjust action probabilities based on known spatial relationships among user interface elements. Such rules may be authored by a system designer, a clinician, a caregiver, or any person with knowledge of the interface functionality and the user's interaction needs, or generated by a machine learning model based on samples of previous user interactions with user interfaces. In some embodiments, the agent may be configured for online learning, where the agent learns and updates its internal parameters based on interactions between the user and the user device 130 in a current session. While various agentic implementations are described separately above herein, parts or all of multiple different implementations may be used by an agent of the control output generator 124.

At block 262, the control output generator 124 generates control data based on the action estimate and the context-aware action determination. The control data may be control data as described above with respect to block 212. To generate the control data, the control output generator 124 may implement an agent-based system to associate a weight value with the output of a neural decoder 122 that generated the action estimate and a neural decoder 122 that generated a context-aware action. In some embodiments, the control output generator 124 may generate the control data based on the determination of an agentic system that analyzes the output of a neural decoder 122 generated from neural signal data 112 without user interface state data 132, and the user interface state data 132 received from the user device 130 as described above. Further, the control output generator 124 may generate the control data based on confidence values associated with the received action estimate and context-aware action. The control output generator 124 may reweight confidence values associated with information received from one or more neural decoders 122A – 122N based on the user interface state data 132.

At block 264, the control output generator 124 transmits the generated control data to the user device 130 as described above with respect to block 214.

At block 266, the signal control unit 120 receives updated user interface state data 132 from the user device 130 as described above with respect to block 216.

### Example User Interface Control Routine

FIG. 3 illustrates example routine 300 for controlling a user interface and displaying a neural feedback indicator according to some embodiments. The routine 300 may begin at block 302 when the user device 130 receives control data (e.g., generated by the signal control unit 120 as described with respect to FIGS. 2A or 2B above herein) from the signal control unit 120. Alternatively, the routine 300 may begin when the user device 130 is initialized, presents a user interface, transmits user interface state data 132 to the signal control unit 120, or receives an indication that a signal control unit 120 has connected to the user device 130 (e.g., via a physical or wireless connection using the network 150).

At block 304, the user device 130 parses the control data 126. Parsing the control data may allow the user device 130 to determine an action to execute based on the control data 126 received from the signal control unit 120. The control data 126 may include a HID report, or other standardized data object, that includes an indication of an action type, a location of an action, an interface element (e.g., a button, scroll bar, cursor, etc.) to which the action applies, a magnitude of an action (e.g., a distance to move a cursor), a direction of an action (e.g., a scroll direction, a direction to move a cursor, etc.), or any other information used by the user device 130 to execute the action indicated by the control data 126. Further, the control data 126 may include a signal quality metric, or other indication of the signal quality of the neural signal data 112 as determined by the signal control unit 120. The signal quality metric may be used to implement a neurofeedback indicator displayed by the user device 130 and described in further detail below with respect to block 308. The signal quality metric may be bounded by an upper and lower limit. For example, the signal quality metric may indicate signal quality information as a number between 0 and 100, 0 and 255, or any other range that may or may not be inclusive of the bounding values. The signal quality metric may be normalized to fit within the bounded range of the neurofeedback indicator.

At block 306, the user device 130 determines the intended action represented by the control data 126 based on the parsed control data 126. To determine the intended action, the user device 130 may access information for a field in the control data 126 indicating the action type. The user device 130 may further access information for fields parsed from the control data 126 indicating how to execute the action. Instructions for how to execute the action may include pressing a selected user interface element, selecting a user interface element, releasing a click from a selected user interface element or cursor, moving a cursor, scrolling a display window in an indicated direction, scrolling a display window an indicated amount or speed, or any other information indicating how an action identified from the control data 126 is to be performed.

At block 308, the user device 130 updates a neural feedback indicator provided to a user. The user device 130 may alter a characteristic of the neurofeedback indicator based on the strength or consistency of the neural signal data 112 as indicated in the control data 126. Consistency may indicate that the same action is associated with a user’s intent over a period of time. In some embodiments, the neurofeedback indicator may be an audible signal provided to the user. For example, the user device 130 may alter the pitch, volume, or frequency of a tone being emitted by the user device 130 (e.g., the speed at which a beeping noise is provided to the user) based on the neural signal strength. In some embodiments, the neurofeedback indicator may be tactile. For example, a vibration strength, vibration pattern, or other tactile characteristic may be adjusted based on the neural signal strength. In some embodiments, the neurofeedback indicator may be a visual indicator provided by a display of the user device 130 to a user. For example, the neurofeedback indicator may be a bar that fills as neural signal strength increases, changes color based on neural signal strength, provides a numerical or text display indicating neural signal strength, or otherwise visually represents neural signal strength for the user. In some embodiments, the neurofeedback indicator may be a combination of modalities, such as visual and audible.

In some embodiments, the user device 130 may adjust an interaction parameter for an element of the user interface based on the received control data 126. As used herein an interaction parameter may include, for example, a threshold time for a user to select a display element (e.g., a display element with which the user can interact such as an application icon or other selectable element displayed in the UI), a scanning speed, element size (e.g., enlarging or shrinking the area of the display used for a display element), or any other parameter for a display element that can be modified by the user device 130. Enabling the user device 130 to adjust an interaction parameter may improve the overall user experience by allowing the user interface to adapt to changes in the user’s ability to interact with the user device 130. For example, when a user is fatigued the user’s ability to control a cursor may be reduced. The user’s fatigue, or otherwise reduced capacity to interact with the user device 130, may be represented in the signal strength information of the control data 126 (e.g., as a low signal strength). In this example, the user device 130 may increase the size of icons displayed in the user interface to adapt to the user’s reduced cursor control by providing larger selection targets.

At block 310, the user device 130 causes presentation of the neural feedback indicator. Causing presentation of the neural feedback indicator may include updating a previously displayed neural feedback indicator based on a current neural signal strength indicated in the received control data 126. Causing display of the neural feedback indicator may include moving the position or adjusting the size of a neural feedback indicator on a screen of the user device 130, or providing an animation displayed to the user. For example, if a currently displayed neural feedback indicator at least partly obscures a user interface element that a user may intend to interact with, the user device 130 may resize or reposition the neural feedback indicator to prevent or minimize the obscuring of that user interface element. Additionally, when a neural feedback indicator reaches an upper signal or confidence bound, the user device 130 may update the neural feedback indicator to indicate that the action is being performed or has been performed.

At decision block 312, the user device 130 determines whether a threshold neural signal strength value has been satisfied. The threshold may be an upper bound of a measured neural signal strength. For example, if neural signal strength values are normalized between 0 and 255, inclusive of the bounding values, the threshold may be satisfied when the user device 130 parses the control data 126 and determines the neural signal strength indicated in the control data 126 has a value of 255. The neural signal strength value may be based at least in part on consistency of the neural signal. For example, the user device 130 may determine whether a same action has been indicated for a threshold period of time. When the same action has been indicated by the control data 126 for the threshold period of time, the threshold may be satisfied. When the threshold is satisfied, the routine 300 may move to block 316 and initiate an indicated action. When the threshold is not satisfied, the routine 300 may move to decision block 314, or to block 318 directly.

At decision block 314, the user device 130 determines whether the user interface context has changed. For example, in a scanning mode, the user device 130 may determine that a new user interface element has been selected due to a change in position of the scanner element. In another example, where the neural feedback indicator has been moved to avoid obscuring a user interface element, the user device 130 may determine the user interface context has changed. Any change to the user interface presented by the user device 130 may indicate a change in the user interface context. Advantageously, in embodiments where user interface state data 132 is not sent when the user interface context has not changed, data transfer between the user device 130 and signal control unit 120 may be reduced. In some embodiments, the routine 300 may skip decision block 314 and proceed directly to block 318. When the user device 130 determines the user interface context has changed, the routine 300 proceeds to block 318. When the user device 130 determines the user interface context has not changed, the routine 300 may return to block 302 or another block of the routine 300.

At block 316, the user device 130 initiates an action indicated in the control data 126 based on the threshold neural signal strength having been satisfied. Initiating the action may cause a change in the information displayed by a user interface of the user device 130. The action may be defined in a most recently received control data 126, or in any control data 126 associated with the neural signal strength having been satisfied. To determine the action to initiate, the user device 130 may analyze a time stamp in the control data 126 received from the signal control unit 120 and compare the time stamp to previously sent user interface state data 132, previously received control data 126, and/or a current time to determine the action associated with the threshold neural signal strength having been satisfied.

At block 318, the user device 130 transmits updated user interface state data 132 to the signal control unit 120. The updated user interface state data 132 may be an absolute representation of the user interface displayed by the user device 130. For example, the user interface state data 132 may include an indication of a current cursor position, button status, position of user interface elements, and any other user interface information. In some embodiments, the user interface state data 132 may be a delta representation of the user interface that indicates changes to the user interface relative to a previously-sent user interface state data 132.

### Example User Interface with Neurofeedback

FIG. 4 illustrates example user interface 400 presented by the user device 130 to a user and controlled based on information received by the user device 130 from the signal control unit 120 (e.g., the control data 126). The user interface 400 of this example includes a first icon 402, a second icon 404, a third icon 406, a cursor 410, and a neurofeedback indicator 420.

The icons 402 – 406 in this example user interface 400 represent applications the user may intend to select using the cursor 410. The second icon 404 is a currently selected icon as indicated by the change in the shape of the bounding box of the second icon 404 from a curved bounding box to a square bounding box. The change in bounding box shape is exemplary, and the currently selected icon may be indicated in various different ways such as displaying or updating a frame around the icon (e.g., applying an additional border or modifying the border of the icon), highlighting the icon, changing a color of the icon, changing a shape of the icon, applying a translucent mask over the icon, or otherwise modifying a display element of the icon to indicate the icon is selected. In some embodiments, the user device 130 may not adjust the display of a currently selected icon. Additionally, the icon 404 shown in this example is being filled by dashed lines as a neurofeedback indicator. The neurofeedback indicator 420 described below may be displayed in addition to, or in place of, including neurofeedback indication in the icon 404. Further, while dashed lines are shown here, various other options may be implemented for displaying neurofeedback using an icon in the user interface 400. For example, the icon 404 may be recolored, filled or highlighted with a color proportional to the neurofeedback value intended to be conveyed to the user, the outline or border of the icon may be progressively filled to provide a neurofeedback indication (e.g., a color may be overlaid on the icon 404 border starting at a position and continuing to cover the border until the entire border is colored to indicate that neurofeedback has reached a threshold value), or displayed in any other manner to convey neurofeedback using the icon 404.

The cursor 410 is a two-dimensional (2D) cursor that can be moved based on control data 126 received from the signal control unit 120 to different positions on the screen. The control data 126 may also include the ability for a user to “click.” In this example user interface 400, the user has positioned the cursor 410 over the selected second icon 404 and initiated a click action. As a result of the user initiating the click action, the user device 130 displays the neurofeedback indicator 420. In some embodiments, the neurofeedback indicator 420 may be displayed even when a user is not initiating a click or other selection (e.g., selecting an icon in a scanning mode) action.

The neurofeedback indicator 420 of this example user interface 400 is a rectangular bar with a set of progress indicators. While a rectangular bar is shown in this example user interface 400, any shape may be used (e.g., a circle, triangle, square, etc.) and the progress indicators may be arranged in various positions depending on the shape used (e.g., if a ring is used progress indicators may be arranged around the circumference of the ring). Further, progress indications may be provided by using the selected display element (e.g., an application icon) itself. For example, the display element may be highlighted, filled in, or have its shape altered to indicate progress in selection The display of the progress indicators, represented here as six portions of the neurofeedback indicator 420 separated by vertical lines, may be adjusted by the user device 130 to represent a signal strength associated with the click action being initiated by the user. The signal strength information may be provided to the user device 130 by the signal control unit 120. In some embodiments, the signal strength may be based at least in part on how long an action indicated in the control data 126 has been received by the user device 130. For example, if the same click action is received by the user device 130, at threshold time intervals the user device 130 may change the display of the neurofeedback indicator 420 to fill the neurofeedback indicator 420.

In this example, when the neurofeedback indicator 420 is filled (e.g., by modifying display of the progress indicators to change from transparent to filled by a color or pattern), the full progress bar indicates the signal strength has reached a threshold value that will cause the click action to occur. While dashed lines are shown to represent filled progress indicators in this neurofeedback indicator 420, this is exemplary only, and the progress indicators may be shown as filled in various different ways. For example, filled progress indicators of the neurofeedback indicator 420 may be shown by changing a color of the progress indicator, a transparency of the progress indicator, or showing a pattern in the progress indicator. In some embodiments, text indicating the action associated with the neurofeedback indicator 420 may be displayed in the user interface 400.

### Execution Environment

FIG. 5 illustrates various components of an example signal control unit 120 configured to implement various functionality described herein.

In some embodiments, the signal control unit 120 may be implemented using any of a variety of computing devices, such as server computing devices, desktop computing devices, personal computing devices, mobile computing devices, mainframe computing devices, midrange computing devices, host computing devices, or some combination thereof. In some embodiments, the signal control unit 120 may be implemented partly or entirely as an implantable device that is implanted in a user’s body (e.g., in connection with the recording device 110).

In some embodiments, the features and services provided by the signal control unit 120 may be implemented as web services consumable via one or more communication networks. In further embodiments, the signal control unit 120 is provided by one or more virtual machines implemented in a hosted computing environment. The hosted computing environment may include one or more rapidly provisioned and released computing resources, such as computing devices, networking devices, and/or storage devices. A hosted computing environment may also be referred to as a “cloud” computing environment.

In some embodiments, as shown, a signal control unit 120 may include: one or more computer processors 502, such as physical central processing units (“CPUs”); one or more network interfaces 504, such as a network interface cards (“NICs”); one or more computer readable medium drives 506, such as a high density disk (“HDDs”), solid state drives (“SSDs”), flash drives, and/or other persistent non-transitory computer readable media; one or more input/output device interfaces 508; and one or more computer-readable memories 510, such as random access memory (“RAM”) and/or other volatile non-transitory computer readable media.

The computer-readable memory 510 may include computer program instructions that one or more computer processors 502 execute and/or data that the one or more computer processors 502 use in order to implement one or more embodiments. For example, the computer-readable memory 510 can store an operating system 512 to provide general administration of the signal control unit 120. As another example, the computer readable memory 510 can store at least one neural decoder 122. As another example, the computer-readable memory 510 can store a control output generator 124.

### Terminology

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware, or combinations of electronic hardware and computer software. To clearly illustrate this interchangeability, various illustrative components, blocks, modules, and steps have been described above generally in terms of their functionality. Whether such functionality is implemented as hardware, or as software that runs on hardware, depends upon the particular application and design conditions imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, "can," "could," "might," "may," “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

Examples of embodiments of the present disclosure can be described in view of the following clauses:

Clause 1. A computer-implemented method for user interface state based neural control using a brain-computer interface, comprising: receiving, from an interactive computing device, user interface (UI) state metadata representing an aspect of a UI of the interactive computing device; capturing neural signals from a user with a brain-computer interface; generating decoder input comprising neural feature data representing features of the neural signals and UI state feature data representing features of the UI state metadata; processing the decoder input to generate a control output using a model configured to jointly condition the control output on the features of the neural signals and the features of the UI state metadata; and transmitting, to the interactive computing device, the control output as a standard input event of the interactive computing device.

Clause 2. The computer-implemented method of clause 1, further comprising generating the neural feature data as a neural feature vector representing a user’s current neural state over a defined time window.

Clause 3. The computer-implemented method of any of clauses 1-2, further comprising generating the UI state feature data as a context embedding representing the aspect of the UI of the interactive computing device.

Clause 4. The computer-implemented method of any of clauses 1-3, wherein generating the decoder input comprises concatenating a neural feature vector and a context embedding.

Clause 5. The computer-implemented method of any of clauses 1-4, wherein receiving the UI state metadata comprises receiving data representing at least one of: an identifier of a currently focused UI element, an identifier of an active application, an interaction modality indicator, pointer position data, or an interaction timing parameter.

Clause 6. A computer-implemented method for user interface state based neural control using a brain-computer interface, comprising: receiving, from an interactive computing device, user interface (UI) state metadata representing an aspect of a UI of the interactive computing device; capturing neural signals from a user with a brain-computer interface; generating, using a decoder model and neural feature data representing features of the neural signals, action probability data representing probabilities for each of a plurality of candidate actions intended by the user; processing the action probability data and the UI state metadata to generate control output representing a candidate action of the plurality of candidate actions; and transmitting, to the interactive computing device, the control output as a standard input event of the interactive computing device to perform the candidate action.

Clause 7. The computer-implemented method of clause 6, wherein processing the action probability data comprises adjusting one or more probabilities in the action probability data based on the UI state metadata.

Clause 8. The computer-implemented method of clause 7, wherein adjusting one or more probabilities of the action probability data comprises applying a model trained to associate UI context states with adjustments to action probabilities.

Clause 9. The computer-implemented method of any of clauses 7-8, wherein adjusting one or more probabilities of the action probability data comprises determining an adjustment using one or more of a rule, heuristic, or lookup table.

Clause 10. A computer-implemented method for context-aware neural control using a brain-computer interface, comprising: capturing neural signals from a user with a brain-computer interface; generating a first control output based on execution of a decoding operation on a first subset of the neural signals; transmitting, to an interactive computing device via a bidirectional human interface device (HID) communication protocol, the first control output as a standard input event of the interactive computing device; receiving, from the interactive computing device via the bidirectional HID communication protocol, user interface (UI) state metadata representing an aspect of a user interface of the interactive computing device; and generating a second control output based on execution of the decoding operation on a second subset of the neural signals and the UI state metadata.

Clause 11. The computer-implemented method of clause 10, wherein receiving the UI state metadata includes receiving at least one of: an identifier of a currently focused UI element, an identifier of an active application, an interaction modality indicator, pointer position data, or an interaction timing parameter.

Clause 12. The computer-implemented method of any of clauses 10-11, wherein generating the second control output comprises automatically selecting between discrete decoding and continuous decoding based on the UI state metadata and without explicit user-initiated mode switching.

Clause 13. The computer-implemented method of any of clauses 10-12, further comprising transmitting, in parallel with at least one control output, a signal quality metric representative of confidence in the decoding.

Clause 14. The computer-implemented method of clause 13, further comprising generating the signal quality metric based on at least one of: a spectral power measured from a neural signal, or a confidence in a control output generated by the decoding operation.

Clause 15. A brain-computer interface system comprising: a neural interface device configured to detect a neural signal from a brain of an individual and to transmit an electronic signal representative of the neural signal; and a signal control unit comprising a decoder communicatively coupled to the neural interface device, the signal control unit configured to: generate a control output based on execution of a decoding operation on the electronic signal; generate a signal quality metric based on at least one of: a spectral power measured from the electronic signal, or a confidence in the control output generated by the decoder; and transmit to an interactive computing device: the control output as a standard input event of the interactive computing device; and the signal quality metric, wherein the interactive computing device is configured to present neurofeedback based on the signal quality metric.

Clause 16. The brain-computer interface system of clause 15, wherein the signal quality metric comprises a value within a range of classification confidence values.

Clause 17. The brain-computer interface system of any of clauses 15-16, wherein the signal quality metric comprises a value within a range of spectral power values.

Clause 18. The brain-computer interface system of any of clauses 15-17, wherein to generate the signal quality metric, the signal control unit is configured to: determine a degree of power in one or more task-relevant neural frequency bands of the electronic signal; determine a degree of confidence in output of the decoder; and generate the signal quality metric based on the degree of power and the degree of confidence.

Clause 19. The brain-computer interface system of any of clauses 15-18, wherein the signal control unit is configured to transmit to the interactive computing device a switch identifier representing a switch to be activated.

Clause 20. The brain-computer interface system of any of clauses 15-19, wherein the signal control unit is configured to continuously evaluate and transmit the signal quality metric to the interactive computing device until the signal quality metric reaches a threshold value, and wherein the interactive computing device is configured to continuously update presentation of the neurofeedback based on receipt of the signal quality metric.

Clause 21. The brain-computer interface system of any of clauses 15-20, wherein the neurofeedback comprises an animation that is continuously updated based on the signal quality metric.

Clause 22. A method for controlling a computing device using neural signals, comprising: detecting neural activity of a user with a brain-computer interface; decoding the neural activity to generate a control output; transmitting the control output to an operating system as a native input event using a human interface device protocol such that the operating system performs a user interface action; receiving, from the operating system and via the human interface device protocol, semantic user interface context information generated by the operating system, the semantic user interface context information including at least one of: (i) an identifier of a currently focused user interface element, (ii) an interaction modality associated with the focused user interface element, or (iii) an interaction timing parameter associated with an accessibility scanning or dwell process; adapting a decoding operation applied to subsequently detected neural activity based at least in part on the received semantic user interface context information, including selecting between discrete decoding and continuous decoding without user-initiated mode switching; and transmitting a subsequent control output to the operating system as a native input event using the human interface device protocol, wherein the adapting is driven by user interface state generated by the operating system rather than inferred solely from neural activity, and

Clause 23. The method of clause 22, further comprising transmitting, concurrently with at least one control output, a signal quality metric representing confidence in the decoded neural activity, the signal quality metric being usable by the operating system to modify an interaction parameter of the user interface in real-time.

Clause 24. A brain-computer interface control system, comprising: a neural signal acquisition component configured to obtain neural signals representing user intent; a decoder configured to convert the neural signals into control outputs for operating a computing device; and a context input interface configured to receive user interface context information from an external operating system, wherein the decoder is configured to adapt at least one decoding parameter for converting the neural signals into the control outputs based at least in part on the received user interface context information.

Clause 25. The brain-computer interface control system of clause 24, wherein the user interface context information includes an identifier of a currently focused user interface element.

Clause 26. The brain-computer interface control system of any of clauses 24-25, wherein the user interface context information includes an indication of whether a target user interface element supports discrete interaction or continuous interaction.

Clause 27. The brain-computer interface control system of any of clauses 24-26, wherein adapting the decoding parameter includes adjusting at least one of a classification threshold, or a timing parameter.

Clause 28. The brain-computer interface control system of any of clauses 24-27, wherein the decoder executes on a computing device separate from a computing device executing the operating system.

Clause 29. A brain-computer interface communication system, comprising: a decoder configured to generate control outputs from neural signals; and a human interface device adapter configured to transmit the control outputs to an external operating system as native input events, wherein the human interface device adapter is further configured to receive semantic user interface metadata from the operating system and to provide the semantic user interface metadata to the decoder.

Clause 30. The brain-computer interface communication system of clause 29, wherein the semantic user interface metadata includes an identifier of a user interface element associated with a native input event.

Clause 31. The brain-computer interface communication system of any of clauses 29-30, wherein the semantic user interface metadata includes interaction timing or state information.

Clause 32. The brain-computer interface communication system of any of clauses 29-31, wherein the human interface device adapter transmits the control outputs and receives the semantic user interface metadata using a standardized human interface device protocol.

Clause 33. The brain-computer interface communication system of any of clauses 29-32, wherein the decoder is configured to modify subsequently generated control outputs based on the received semantic user interface metadata.

Clause 34. A brain-computer interface system, comprising: a neural decoder configured to generate control outputs from neural signals; a confidence evaluation module configured to generate a signal-quality metric associated with the control outputs; and a communication interface configured to transmit the control outputs and the signal-quality metric to an external operating system.

Clause 35. The brain-computer interface system of clause 34, wherein the signal-quality metric represents confidence, strength, or reliability of the decoded neural signals.

Clause 36. The brain-computer interface system of any of clauses 34-35, wherein the signal-quality metric is transmitted concurrently with a corresponding control output.

Clause 37. The brain-computer interface system of any of clauses 34-36, wherein the signal-quality metric is continuously updated during user interaction.

Clause 38. The brain-computer interface system of any of clauses 34-37, wherein the signal-quality metric is configured to enable dynamic modification of user interaction behavior by the operating system.

Clause 39. A brain-computer interface control system, comprising: a neural signal decoder configured to generate both discrete control outputs and continuous control outputs from neural signals; and a control interface configured to transmit the discrete control outputs and the continuous control outputs to an external operating system as native input events.

Clause 40. The brain-computer interface control system of clause 39, wherein the discrete control outputs correspond to selection or activation actions.

Clause 41. The brain-computer interface control system of any of clauses 39-40, wherein the continuous control outputs include directional and magnitude-based pointer movement.

Clause 42. The brain-computer interface control system of any of clauses 39-41, wherein selection between discrete control outputs and continuous control outputs is based on context information received from the operating system.

Clause 43. The brain-computer interface control system of any of clauses 39-42, wherein the discrete control outputs and the continuous control outputs are generated by the same decoder without requiring an explicit mode selection by a user.

Clause 44. A distributed brain-computer interface system, comprising: a neural signal decoder executing on a first computing device; a communication interface configured to transmit control outputs from the decoder to an external operating system executing on a second computing device; and a feedback interface configured to receive user interface context information from the operating system, wherein the decoder is configured to adapt generation of the control outputs based at least in part on the received user interface context information.

Clause 45. The distributed brain-computer interface system of clause 44, wherein the first computing device is physically separate from the second computing device.

Clause 46. The distributed brain-computer interface system of any of clauses 44-45, wherein the decoder is configured to control multiple external operating systems.

Clause 47. The distributed brain-computer interface system of any of clauses 44-46, wherein the communication interface comprises a wireless communication interface.

Clause 48. The distributed brain-computer interface system of any of clauses 44-47, wherein the decoder maintains state information across interactions with different operating systems.

What is claimed is:

1. A computer-implemented method for user interface state based neural control using a brain–computer interface, comprising:
receiving, from an interactive computing device, user interface (UI) state metadata representing an aspect of a UI of the interactive computing device;
capturing neural signals from a user with a brain–computer interface;
generating decoder input comprising neural feature data representing features of the neural signals and UI state feature data representing features of the UI state metadata;
processing the decoder input to generate a control output using a model configured to jointly condition the control output on the features of the neural signals and the features of the UI state metadata; and
transmitting, to the interactive computing device, the control output as a standard input event of the interactive computing device.

2. The computer-implemented method of claim 1, further comprising generating the neural feature data as a neural feature vector representing a user’s current neural state over a defined time window.

3. The computer-implemented method of claim 1, further comprising generating the UI state feature data as a context embedding representing the aspect of the UI of the interactive computing device.

4. The computer-implemented method of claim 1, wherein generating the decoder input comprises concatenating a neural feature vector and a context embedding.

5. The computer-implemented method of claim 1, wherein receiving the UI state metadata comprises receiving data representing at least one of: an identifier of a currently focused UI element, an identifier of an active application, an interaction modality indicator, pointer position data, or an interaction timing parameter.

6. A computer-implemented method for user interface state based neural control using a brain–computer interface, comprising:
receiving, from an interactive computing device, user interface (UI) state metadata representing an aspect of a UI of the interactive computing device;
capturing neural signals from a user with a brain–computer interface;
generating, using a decoder model and neural feature data representing features of the neural signals, action probability data representing probabilities for each of a plurality of candidate actions intended by the user;
processing the action probability data and the UI state metadata to generate control output representing a candidate action of the plurality of candidate actions; and
transmitting, to the interactive computing device, the control output as a standard input event of the interactive computing device to perform the candidate action.

7. The computer-implemented method of claim 6, wherein processing the action probability data comprises adjusting one or more probabilities in the action probability data based on the UI state metadata.

8. The computer-implemented method of claim 7, wherein adjusting one or more probabilities of the action probability data comprises applying a model trained to associate UI context states with adjustments to action probabilities.

9. The computer-implemented method of claim 7, wherein adjusting one or more probabilities of the action probability data comprises determining an adjustment using one or more of a rule, heuristic, or lookup table.

10. A computer-implemented method for context-aware neural control using a brain–computer interface, comprising:
capturing neural signals from a user with a brain–computer interface;
generating a first control output based on execution of a decoding operation on a first subset of the neural signals;
transmitting, to an interactive computing device via a bidirectional human interface device (HID) communication protocol, the first control output as a standard input event of the interactive computing device;
receiving, from the interactive computing device via the bidirectional HID communication protocol, user interface (UI) state metadata representing an aspect of a user interface of the interactive computing device; and
generating a second control output based on execution of the decoding operation on a second subset of the neural signals and the UI state metadata.

11. The computer-implemented method of claim 10, wherein receiving the UI state metadata includes receiving at least one of: an identifier of a currently focused UI element, an identifier of an active application, an interaction modality indicator, pointer position data, or an interaction timing parameter.

12. The computer-implemented method of claim 10, wherein generating the second control output comprises automatically selecting between discrete decoding and continuous decoding based on the UI state metadata and without explicit user-initiated mode switching.

13. The computer-implemented method of claim 10, further comprising transmitting, in parallel with at least one control output, a signal quality metric representative of confidence in the decoding.

14. The computer-implemented method of claim 13, further comprising generating the signal quality metric based on at least one of: a spectral power measured from a neural signal, or a confidence in a control output generated by the decoding operation.

15. A brain-computer interface system comprising:
a neural interface device configured to detect a neural signal from a brain of an individual and to transmit an electronic signal representative of the neural signal; and
a signal control unit comprising a decoder communicatively coupled to the neural interface device, the signal control unit configured to:
generate a control output based on execution of a decoding operation on the electronic signal;
generate a signal quality metric based on at least one of: a spectral power measured from the electronic signal, or a confidence in the control output generated by the decoder; and
transmit to an interactive computing device:
the control output as a standard input event of the interactive computing device; and
the signal quality metric, wherein the interactive computing device is configured to present neurofeedback based on the signal quality metric.

16. The brain-computer interface system of claim 15, wherein the signal quality metric comprises a value within a range of classification confidence values.

17. The brain-computer interface system of claim 15, wherein the signal quality metric comprises a value within a range of spectral power values.

18. The brain-computer interface system of claim 15, wherein to generate the signal quality metric, the signal control unit is configured to:
determine a degree of power in one or more task-relevant neural frequency bands of the electronic signal;
determine a degree of confidence in output of the decoder; and
generate the signal quality metric based on the degree of power and the degree of confidence.

19. The brain-computer interface system of claim 15, wherein the signal control unit is configured to transmit to the interactive computing device a switch identifier representing a switch to be activated.

20. The brain-computer interface system of claim 15, wherein the signal control unit is configured to continuously evaluate and transmit the signal quality metric to the interactive computing device until the signal quality metric reaches a threshold value, and wherein the interactive computing device is configured to continuously update presentation of the neurofeedback based on receipt of the signal quality metric.
